(19) United States
(12) Patent Application Publication
(10) Pub. No.: US 2007/0189987 A1
(43) Pub. Date: Aug. 16, 2007

(54) Composition comprising a monomeric compound with an optical effect, process using said composition, monomeric compound, polymer comprising the same and use thereof

(76) Inventor: Timo Luukas, Massy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/592,050

(22) PCT Filed: Feb. 28, 2005
(86) PCT No.: PCT/FR05/00475

§ 371 Date: Jan. 26, 2007

Related U.S. Application Data
(60) Provisional application No. 60/560,617, filed on Apr. 9, 2004.
(60) Provisional application No. 60/560,705, filed on Apr. 9, 2004.

(30) Foreign Application Priority Data
Mar. 9, 2004 (FR) 0450475
Mar. 9, 2004 (FR) 0450474

(51) Int. Cl. A61K 8/81; A61Q 1/06; A61Q 1/08; A61Q 1/12; A61Q 17/04; A61Q 19/04; A61Q 19/08; A61Q 5/02; A61Q 5/06; C07D 221/04

(52) U.S. Cl. 424/59; 424/61; 424/63; 424/64; 424/69; 424/70.1; 424/70.7; 546/98

(57) ABSTRACT

The invention relates to a cosmetic or pharmaceutical composition containing, in a physiologically-acceptable medium, at least one polymer comprising at least one novel monomeric compound having optical effect properties. The invention also relates to a cosmetic method of making up or caring for keratin materials, such as the skin belonging to the body or face, lips, nails, eyelashes, eyebrows and/or hair, consisting in applying one such cosmetic composition to said materials. The invention further relates to novel monomeric compounds having formula (I) and optical properties, the polymers comprising same and the uses thereof in a composition in order to provide said composition with optical effects, e.g. fluorescence or optical brightening.

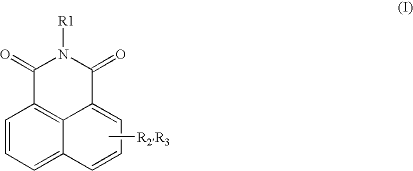

The present invention relates to novel cosmetic or pharmaceutical compositions, especially for topical application, and especially to novel makeup compositions, comprising organic polymers with particular optical properties and especially fluorescence properties. The present invention also relates to novel monomeric compounds with optical properties, especially fluorescence properties, and also to polymers that may be prepared from these compounds.

Cosmetic compositions, and especially makeup compositions such as loose or compact powders, foundations, makeup rouges, eyeshadows, lipsticks or nail varnishes, generally consist of a suitable vehicle and one or more coloring agents intended to give said compositions a certain color before and/or after applying them to the skin, mucous membranes, semimucous membranes and/or the integuments such as the nails, the eyelashes or the hair.

To create colors, a fairly limited range of coloring agents is used at the present time, especially comprising lakes, mineral pigments, organic pigments and nacreous pigments.

The pigments and lakes used in the makeup field are of very diverse origin and chemical nature. Their physico-chemical properties, especially their granulometry, specific surface area, density, etc., are thus very different. These differences are reflected by variations in behavior: their ease of use or of dispersion in the medium; their light and heat stability; their mechanical properties. Mineral pigments, in particular mineral oxides, are, on the other hand, very stable to light and to pH, but give rather dull, pale colors. It is thus necessary to introduce a large amount of them into cosmetic formulations in order to obtain a sufficiently saturated mark. This high percentage of mineral particles can, however, affect the gloss of the composition. As regards nacreous pigments, they can produce varied colors, but of relatively weak intensity, which lead to iridescent effects that are usually quite weak. In the field of temporary or short-term hair dyeing, which gives rise to a slight change in the natural color of the hair that holds from one shampoo wash to the next and that serves to enhance or correct an already-established shade, coloration with common pigments to give the hair a temporary tint has already been proposed, but the shades obtained by this coloration remain quite dull, too uniform and rather boring.

In the field of makeup, only organic lakes have made it possible until now to obtain bright, vivid colors. However, most organic lakes have very poor lightfastness, which is reflected by pronounced attenuation of their color over time. They may also be heat- and/or pH-unstable. Furthermore, certain lakes produce excessive bleeding, i.e. they have the drawback of staining the support onto which they are applied. Thus, this may have the consequence of staining ocular lenses in the case of eyeliners or mascaras, or of leaving a coloration on the skin or the nails after makeup removal in the case of lipsticks or nail varnishes. Finally, the instability of lakes is also worsened when they are combined with photoreactive pigments, for instance titanium dioxide. Now, as it happens, these pigments are very widely used in makeup, especially for protecting against UV radiation. Consequently, the use of organic lakes in cosmetics is quite limited, which has the consequence of limiting the shades that may be produced.

Thus, there is still a need for organic polymers with optical properties, which can be used in cosmetics, to give compositions comprising them and/or makeup obtained using these compositions adequate optical effects, said polymers moreover having good heat and photochemical stability, while at the same time producing little bleeding.

After considerable research, the Applicant has demonstrated that the use of a specific family of polymers, in fact comprising at least one specific monomer, unexpectedly allows such a result to be obtained.

Thus, one subject of the invention is a cosmetic or pharmaceutical composition comprising, in a physiologically acceptable medium, at least one polymer comprising at least one monomeric compound as defined below.

Another subject of the invention is a cosmetic process for making up or caring for keratin materials, especially bodily or facial skin, the lips, the nails, the eyelashes, the eyebrows and/or the hair, comprising the application of such a cosmetic composition to said materials.

The polymers according to the invention may be in solid or liquid form and give noteworthy optical effects to the compositions comprising them and also to the applied makeup; in particular, they can give lightening or color effects.

These optical effects may be advantageously modified as a function of the chemical nature and/or the position of the various substitutents present on the monomer with an optical effect used to form the polymer. In general, when the group X is an oxygen, the monomer, and the resulting polymer, will rather be of blue/white color; when the group X comprises a nitrogen atom, the monomer, and the resulting polymer, will rather be in the orange range.

Among the other advantages that the polymers according to the invention can afford, mention may be made of their good heat, pH and light stability.

It has also been found that the polymers according to the invention show good solubility in fatty substances, which can vary and be adjusted according to the nature of the monomers. This good liposolubility can also facilitate their subsequent use, especially in cosmetic compositions generally comprising a fatty phase.

Furthermore, the good cosmetic properties of the compositions according to the invention are maintained when they comprise the polymers according to the invention.

Furthermore, although being of similar chemical structure, the polymers according to the invention may show, depending on the nature of the substitutents, a wide variety of optical effects, which may range from blue/violet to orange/red, passing through yellow. This gives access to a range of compounds, belonging to the same chemical family, and thus being formulated in a similar manner, which offer noteworthy diversities of colors or of optical properties; this especially facilitates the task of formulators by allowing them to keep a common architecture for all of their compositions, irrespective of the polymers with optical properties used.

Moreover, the polymers used according to the invention have good fluorescence properties, and some of them have good optical-brightening properties. It is recalled that optical brighteners are endowed with fluorescence properties; in general, fluorescent compounds absorb in the ultraviolet and visible range, and re-emit energy by fluorescence at a wavelength of between 380 nm and 830 nm; when this wavelength is between 380 nm and 480 nm, i.e. in the blue region of the visible range, the compounds are then optical brighteners.

In addition, the polymers according to the invention have the advantage of undergoing makeup removal more easily than the optical brightening or fluorescent monomolecular compounds of the prior art, such as 2,5-bis(tert-butyl-1,3-benzoxazole)thiophene, especially the product known under the name Uvitex OB.

The composition according to the invention thus comprises, in a physiologically acceptable medium and especially a cosmetically or pharmaceutically acceptable medium, at least one polymer that can be obtained by polymerization, especially free-radical polymerization, of at least one monomer of formula (I).

Said monomer of formula (I) thus corresponds to the following formula: 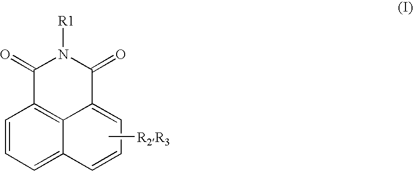
in which:
R1 represents a linear, branched and/or cyclic, saturated and/or unsaturated carbon-based radical containing 1 to 32 carbon atoms; optionally substituted with one or more groups chosen from ═O, OH, NH₂ and halogen atoms; and/or optionally interrupted with one or more heteroatoms chosen from O, N, P, Si and S;
R₂ and R₃, which are present on the same ring or each on a different ring, represent, independently of each other, a hydrogen, a halogen or a group of formula —X-G-P (II), with the proviso that at least one of the radicals R₂ and/or R₃ represents a group of formula (II), in which:
X is chosen from the groups —O—, —S—, —SO—, —SO₂—, —NH— and —NR₄— with R₄ representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
G is a linear, branched and/or cyclic, saturated and/or unsaturated divalent carbon-based radical containing 1 to 32 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae: 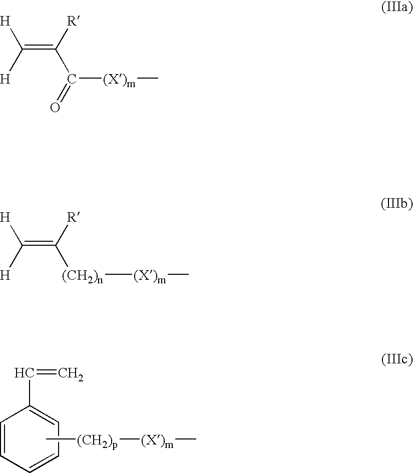
   in which:
R′ represents H or a linear or branched, saturated C₁₋₆ hydrocarbon-based radical,
X′ represents O, NH or NR″ with R″ representing a radical chosen from C₁₋₆ alkyl, C₆₋₁₀ aryl, (C₆₋₁₀)-aryl(C₁₋₆)alkyl and (C₁₋₆)alkyl(C₆₋₁₀)aryl radicals, the alkyl and/or aryl groups also possibly being substituted with one or more groups chosen from OH, halogen, C₁₋₆ alkoxy and C₆₋₁₀ aryloxy;
   and
m is equal to 0 or 1; n is equal to 0 or 1 and p is equal to 0, 1 or 2.

It will be noted that some of the compounds of formula (I) for which, simultaneously, R₂ is H, X is NH, P is of formula (IIIa), m=1, R′ is H or CH₃, X′ is O or NR″ and R1 is a C₁-C₆ alkyl radical, a C₆-C₁₀ aryl radical, a (C₆-C₁₀)aryl(C₁-C₆)alkyl radical or a (C₁-C₆)alkyl(C₆-C₁₀)aryl radical, the alkyl and/or aryl radicals being optionally substituted with a hydroxyl, a halogen, a (C₁-C₆)alkoxy or a (C₆-C₁₀)aryloxy, may be known.

In the present invention, the term “cyclic radical” means a monocyclic or polycyclic radical, which is thus itself in the form of one or more saturated and/or unsaturated, optionally substituted rings (for example cyclohexyl, cyclodecyl, benzyl or fluorenyl), but also a radical that comprises one or more of said rings (for example p-tert-butylcyclohexyl or 4-hydroxybenzyl).

In the present invention, the term “saturated and/or unsaturated radical” means totally saturated radicals, totally unsaturated radicals, including aromatic radicals, and also radicals comprising one or more double and/or triple bonds, the rest of the bonds being single bonds.

Preferably, R1 may especially be a linear and/or branched, saturated or unsaturated cyclic hydrocarbon-based radical, optionally comprising a hydrocarbon-based ring which is itself saturated or unsaturated, containing 3 to 18 and especially 4 to 14 carbon atoms, and may comprise at least one heteroatom, especially one or two nitrogen, oxygen or silicon atoms.

Preferentially, R1 may be a linear, branched and/or cyclic, saturated or unsaturated hydrocarbon-based radical containing 6 to 13 carbon atoms.

R1 may especially be an n-propyl, isopropyl, n-butyl, isobutyl, tert-butyl, n-pentyl, cyclopentyl, n-hexyl, cyclohexyl, n-heptyl, n-octyl, cyclooctyl, decyl, cyclodecyl, dodecyl, cyclododecyl, p-tert-butylcyclohexyl, benzyl or phenyl radical or a radical chosen from ethyl-2-N-pyrrolidine, (2-methyl)-1-ethyl-pyrrolidine, 3-propyltriethoxysilane, dialkyl-pyrimidines and especially 2-(4,6-dimethyl)pyrimidine, the benzothiazyl radical and the fluorenyl radical.

| | |
|---|---|
| 2-methyl-1-ethylpyrrolidine | 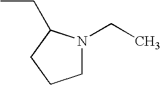 |
| ethyl-2-N-pyrrolidine | 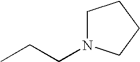 |
| 3-propyltriethoxysilane | 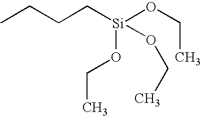 |
| benzothiazyl | 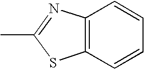 |
| fluorenyl | 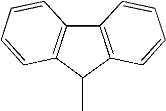 |
| 2-(4,6-dimethyl)pyrimidine | 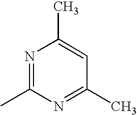 |

The radical R₂ is preferably a hydrogen atom, and R₃ is thus a group of formula (II).

In said group of formula (II), X is preferably chosen from —O—, —S—, —NH— and —NR₄—.

The radical R₄ preferentially represents a linear, branched and/or cyclic, saturated or unsaturated hydrocarbon-based radical optionally comprising a hydrocarbon-based ring that is itself saturated or unsaturated, containing 2 to 18 and especially 3 to 12 carbon atoms, optionally substituted with one or more groups chosen from ═O, OH, NH₂ and halogen atoms; and/or optionally interrupted with one or more heteroatoms chosen from O, N, P, Si and S.

R4 may especially be an ethyl, n-propyl, isopropyl, n-butyl, isobutyl, tert-butyl, pentyl, hexyl, cyclo-hexyl, octyl, decyl, dodecyl, phenyl or benzyl radical.

The divalent radical G is preferably a linear, branched and/or cyclic, saturated or unsaturated divalent hydrocarbon-based radical optionally comprising a hydrocarbon-based ring that is itself saturated or unsaturated, containing in total 2 to 18 and especially 3 to 10 carbon atoms, optionally substituted with one or more groups chosen from ═O, OH, NH₂ and halogen atoms; and/or optionally interrupted with one or more heteroatoms chosen from O, N, P and Si.

Preferentially, G is chosen from linear or branched, saturated divalent hydrocarbon-based radicals optionally comprising a saturated hydrocarbon-based ring, containing in total 2 to 18 and especially 3 to 10 carbon atoms.

Thus, G may be chosen from ethylene, n-propylene, isopropylene (or 1-methylethylene and 2-methyl-ethylene), n-butylene, isobutylene, pentylene, especially n-pentylene, hexylene, especially n-hexylene, heptylene, cyclohexylene, octylene, decylene, cyclohexyldimethylene especially of formula —CH₂—C₆H₁₀—CH₂—, and dodecylene radicals.

In formula (IIIb), if n=0, then, preferably, m=0.

The polymerizable group P is preferably chosen from one of the following formulae: 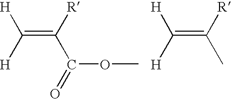
in which R′ represents H or methyl.

Among the monomeric compounds that are particularly preferred according to the invention, mention may be made of the compounds corresponding to one of the following formulae:

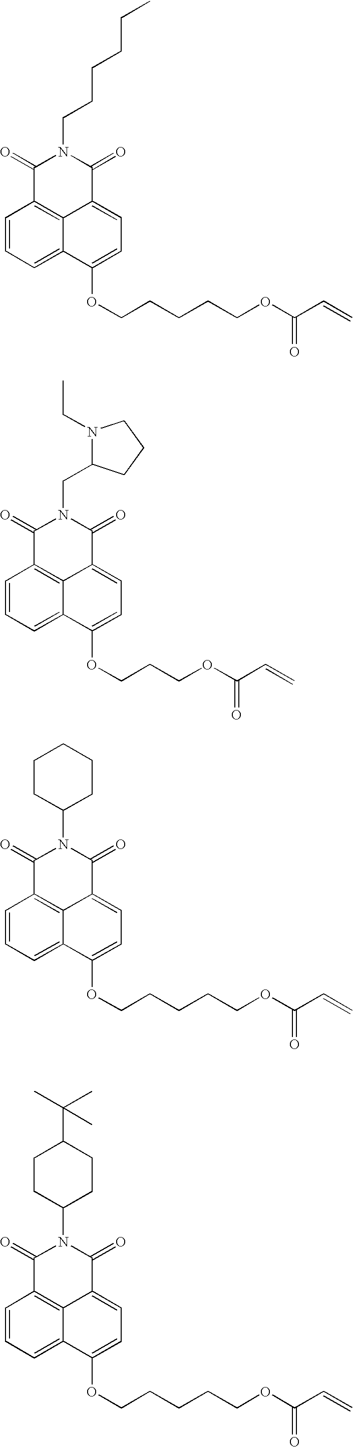

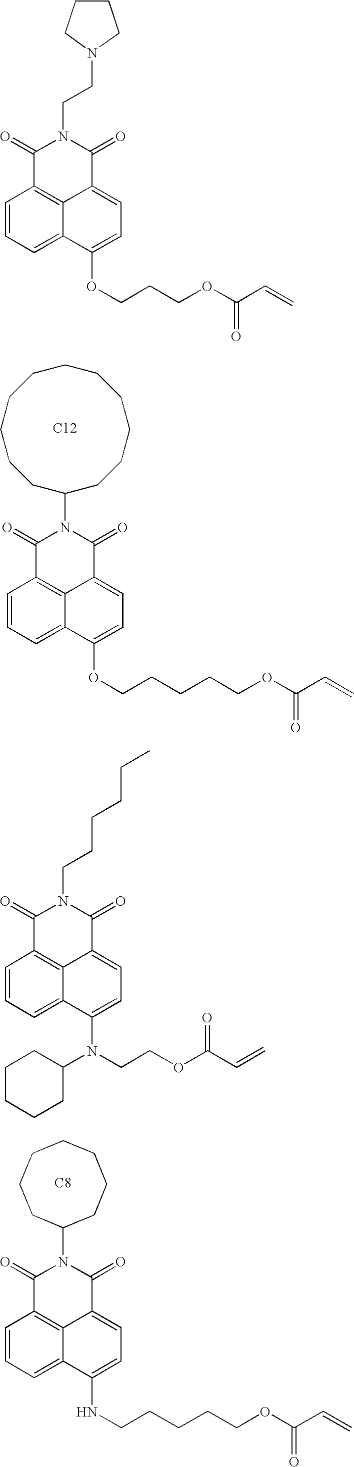

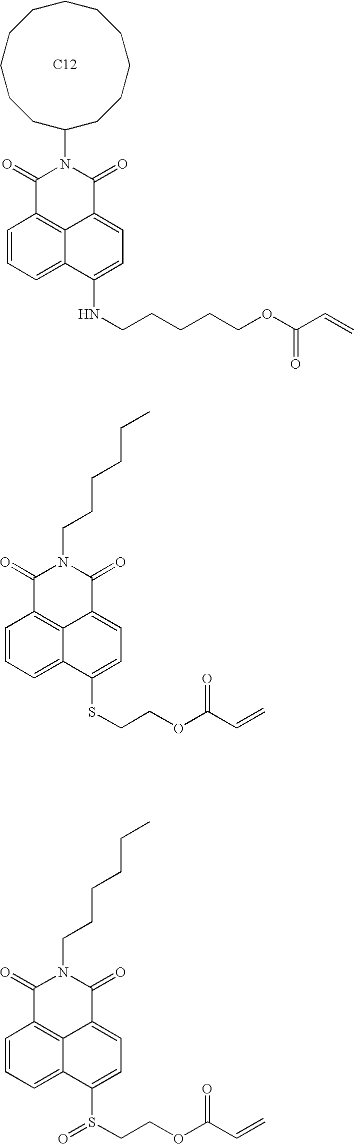

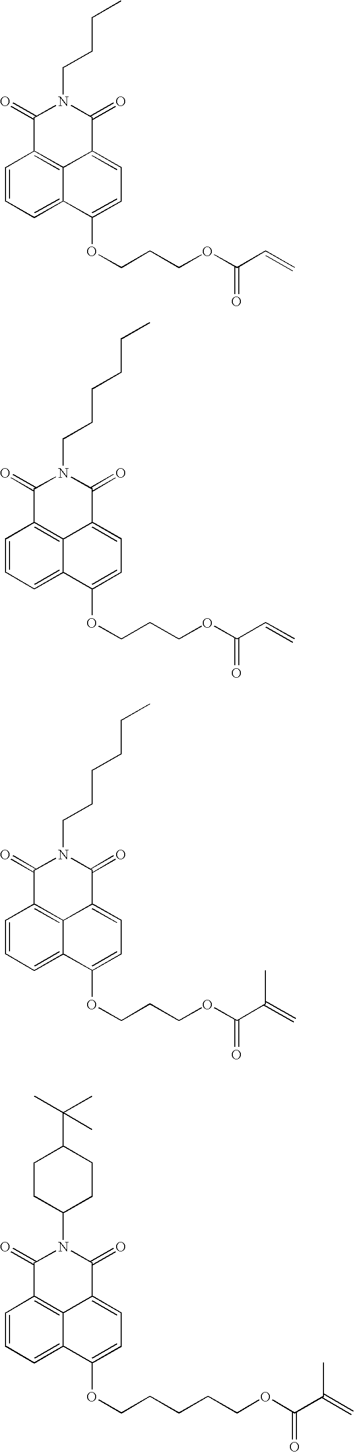

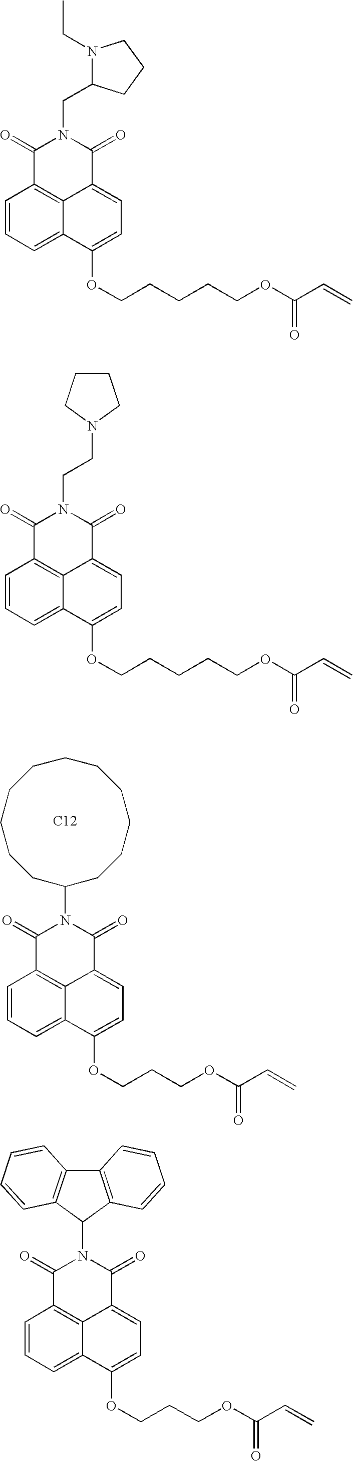

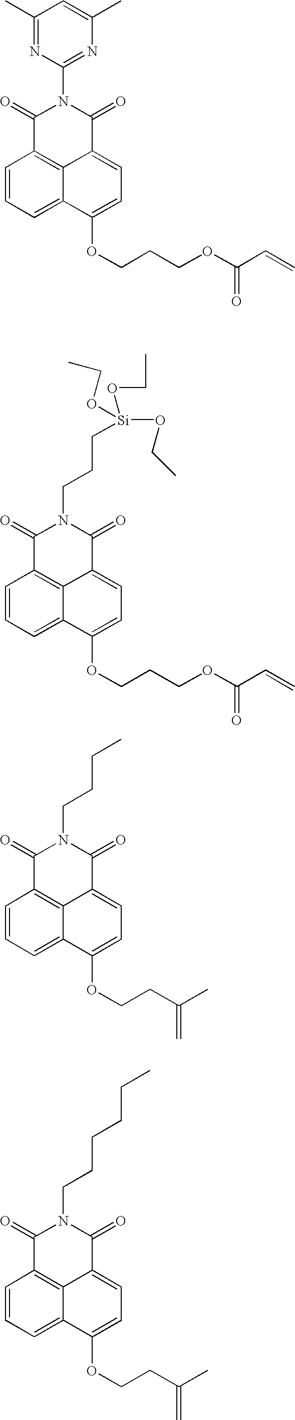

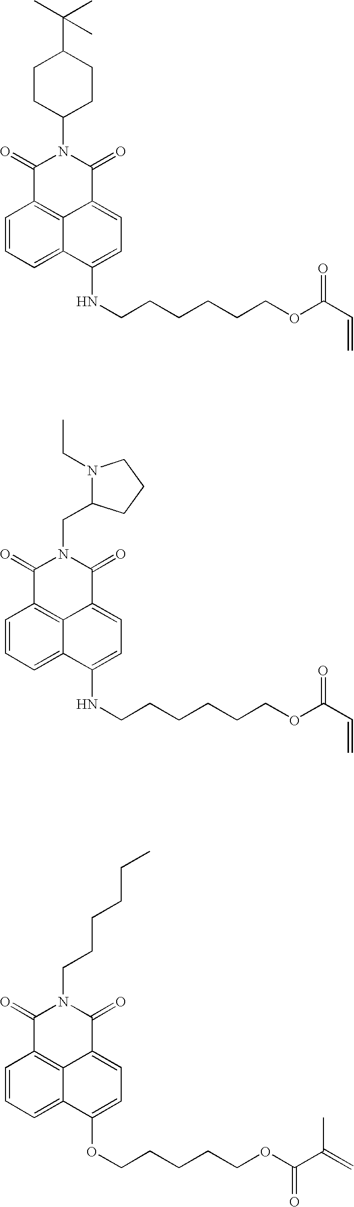

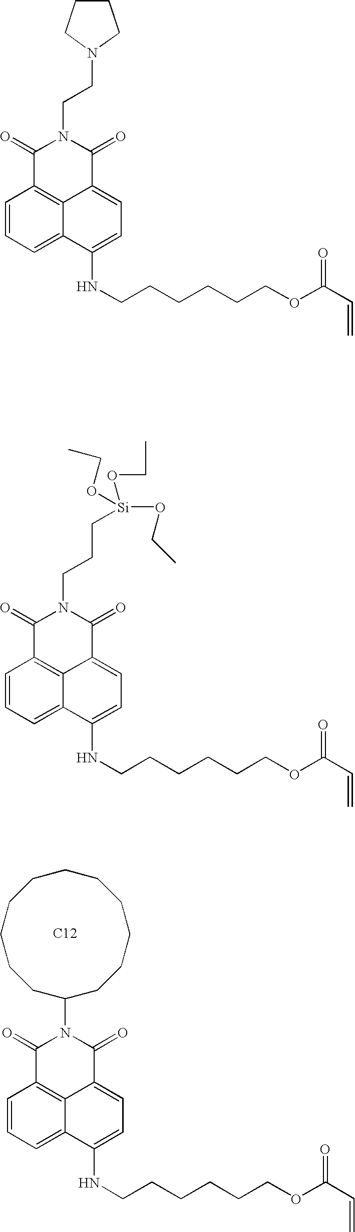

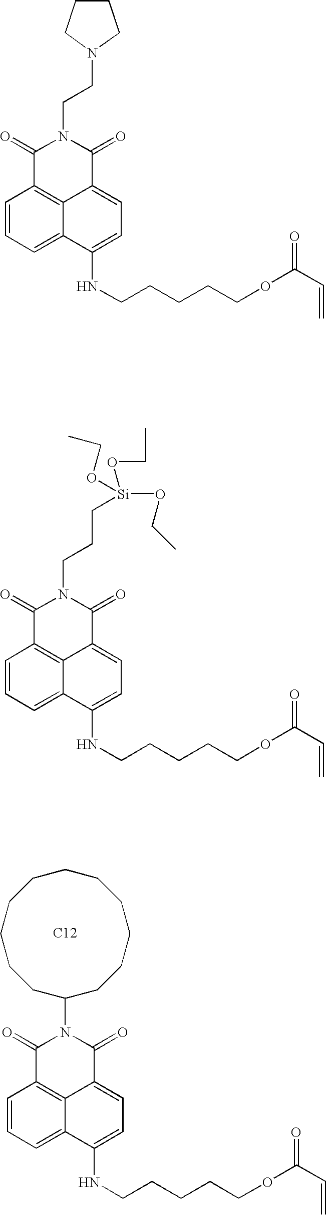

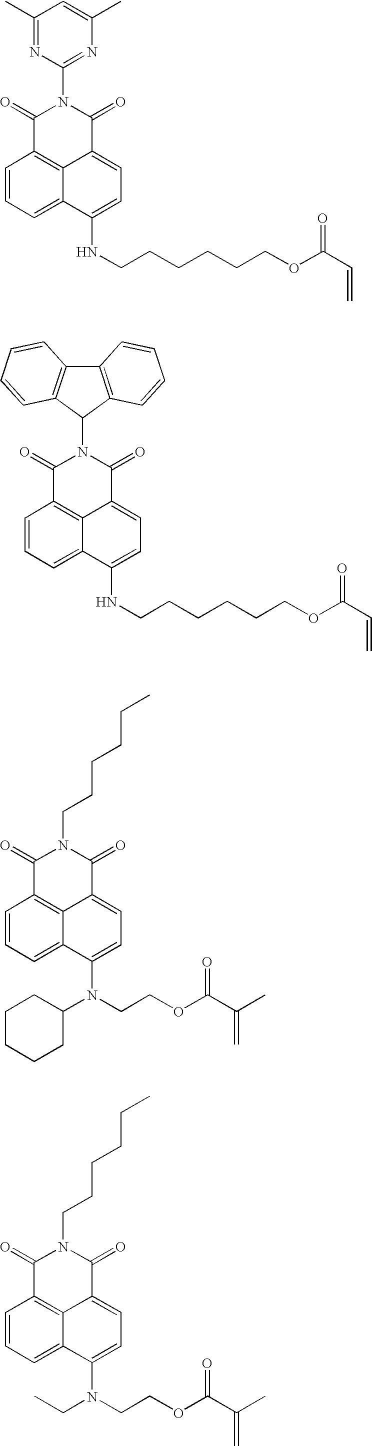

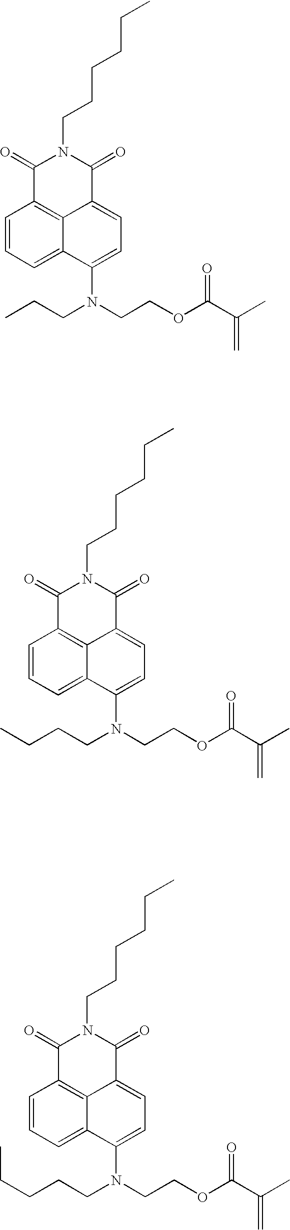

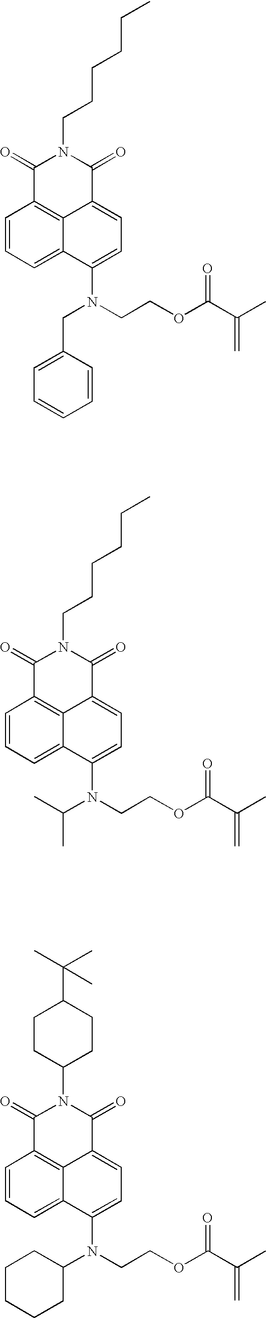

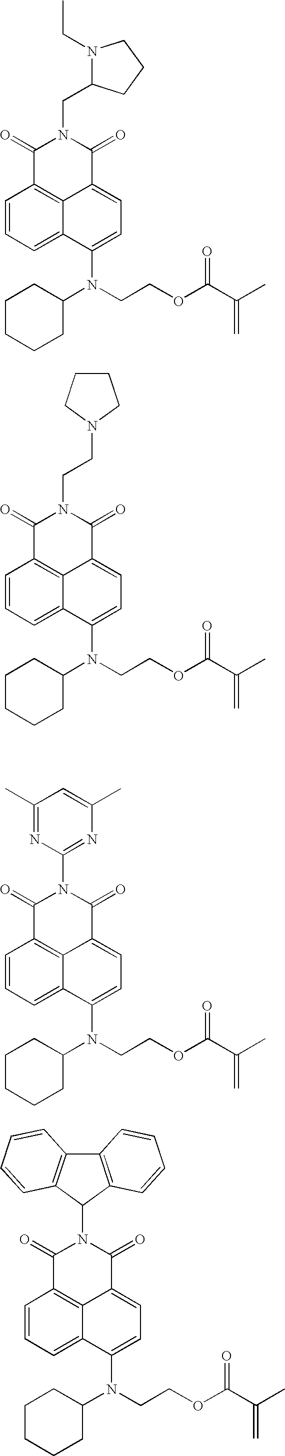

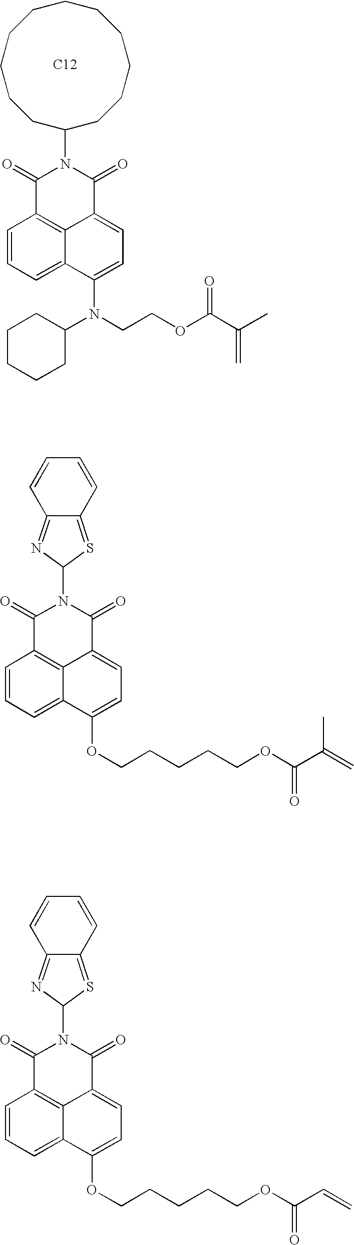

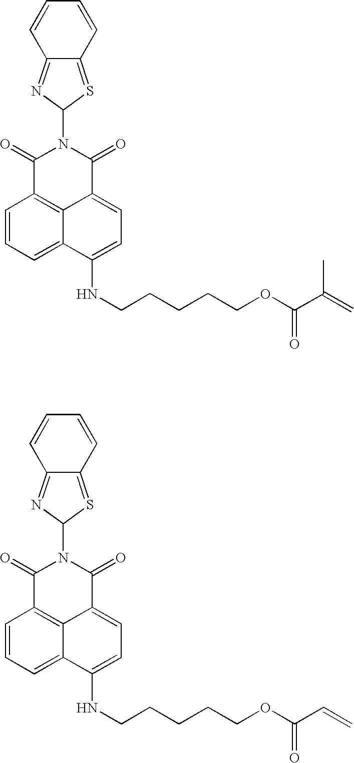

Another subject of the invention is a monomeric compound of formula (I) as defined below: 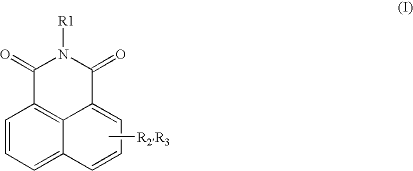
in which:
R1 represents a linear, branched and/or cyclic, saturated and/or unsaturated carbon-based radical, preferably alkyl, containing 2 to 22 carbon atoms and especially 8 to 16 carbon atoms; optionally substituted with one or more groups chosen from ═O, OH, NH₂ and halogen atoms; and/or optionally interrupted with one or more heteroatoms chosen from O, N, P, Si and S;
R₂ and R₃, present on the same ring or each on a different ring, represent, independently of each other, a hydrogen, a halogen or a group of formula —X-G-P (II), with the proviso that at least one of the radicals R₂ and/or R₃ represents a group of formula (II), in which:
X is chosen from —O—, —S—, —SO—, —SO₂—, —NH— and —NR₄— groups with R₄ representing a linear, branched and/or cyclic, saturated and/or unsaturated carbon-based radical containing 2 to 30 carbon atoms, optionally substituted with one or more groups chosen from ═O, OH, NH₂ and halogen atoms; and/or optionally interrupted with one or more heteroatoms chosen from O, N, P, Si and S;
G is a linear, branched and/or cyclic, saturated and/or unsaturated divalent carbon-based radical, containing 2 to 32 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae: 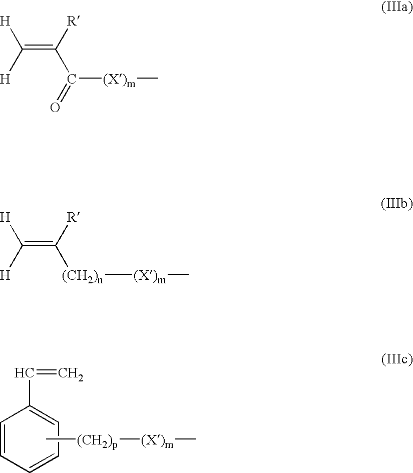
   in which:
R′ represents H or a linear or branched, saturated C₁₋₆ hydrocarbon-based radical,
X′ represents O, NH or NR″ with R″ representing a radical chosen from C₁₋₆ alkyl, C₆₋₁₀ aryl, (C₆₋₁₀)-aryl(C₁₋₆)alkyl and (C₁₋₆)alkyl(C₆₋₁₀)aryl radicals, the alkyl and/or aryl groups also possibly being substituted with one or more groups chosen from OH, halogen, C₁₋₆ alkoxy and C₆₋₁₀ aryloxy;
m is equal to 0 or 1;
n is equal to 0 or 1;
p is equal to 0, 1 or 2,
   with the exclusion of the compounds of formula (I) for which, simultaneously, R₂ is H, X is NH, P is of formula (IIIa), m=1, R′ is H or CH₃, X′ is O or NR″ and R₁ is a C₁-C₆ alkyl radical, a C₆-C₁₀ aryl radical, a (C₆-C₁₀)aryl(C₁-C₆)alkyl radical or a (C₁-C₆)alkyl(C₆-C₁₀)aryl radical, said alkyl and/or aryl radicals being optionally substituted with a hydroxyl, a halogen, a (C₁-C₆)alkoxy or a (C₆-C₁₀)aryloxy.

Another subject of the invention is a polymer comprising at least one such monomeric compound.

Another subject of the invention is the use of such a monomeric compound or of such a polymer comprising it, in a composition, for giving said composition optical effects, especially fluorescence or optical-brightening effects.

The novel monomers, and the polymers comprising them, have good optical properties and can be prepared more easily than those of the prior art.

For the purpose especially of industrial exploitation, monomeric and polymeric compounds of high reactivity are sought, which allows a short reaction (polymerization) time.

Monomers and polymers that have good optical properties, with a wide color range, and which can be used in cosmetics are also sought.

It is found that with the compounds according to the present invention, the polymerization is easier, especially on account of the presence of a spacer group (G).

In addition, the polymers and the monomeric compounds according to the invention find a most particular use for giving a composition optical effects, especially fluorescence or optical-brightening effects.

Some of these compounds may especially be prepared according to the prior art, for example according to the teaching of document EP 728 745, in particular the compounds for which X is N.

Schematically, the general synthetic process, for the compounds for which X is O, may be represented as follows:

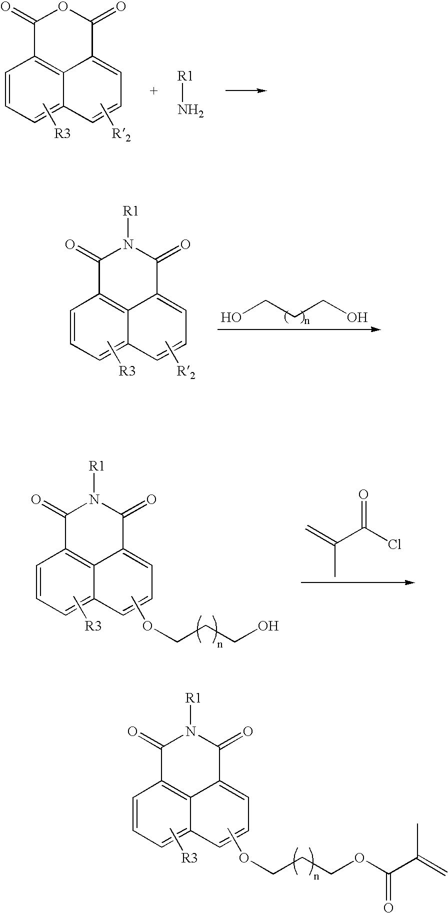

The appropriate naphthalic anhydride may thus be reacted with an appropriate primary amine R1-NH₂.

Preferably, the naphthalic anhydride is present in slight excess relative to the amine, especially in a proportion of from 1 to 1.5 equivalents and preferably 1.1 equivalents per 1 equivalent of amine.

The reaction may be performed in a solvent chosen from solvents in which the anhydride is soluble, and especially toluene, xylene, acetic acid, NMP or ethanol; the reaction is preferably performed at the reflux temperature of the solvent, for example a temperature of 50-250° C. and preferably 75-150° C.

The imide formed can then be reacted with a diol, an amino alcohol or a thio alcohol.

For example, when R′₂ is a halogen (preferably chlorine or bromine), it is possible to perform an aromatic nucleophilic substitution, for example using a diol such as 1,3-propanediol or 1,5-propanediol, optionally in alkali metal (for example sodium) alkoxide form.

The reaction may be performed in the absence of solvent, or in the presence of a dipolar aprotic solvent such as dichloromethane or THF (tetrahydro-furan), especially at a temperature of 20-150° C.

The corresponding alcohol derivative is then obtained, which can then be reacted with a (meth)acryloyl halide, especially a chloride, so as to form the corresponding (meth)acrylate.

This reaction may be performed in the presence of a base such as triethanolamine, in a solvent such as tetrahydrofuran or dichloromethane, especially at a temperature of from −30° C. to 100° C. and preferably 0 to 60° C.

The monomeric compounds for which X is S may be prepared in a similar manner. It is especially possible to react the imide formed in the first step above with a thio alcohol alkali metal alkoxide so as to form the alcoholic derivative according to the scheme below:

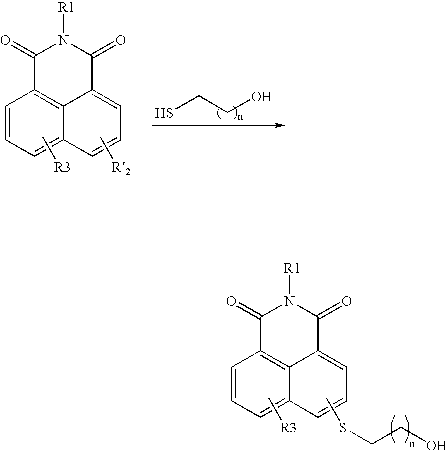

This derivative may then be oxidized under mild conditions so as to give the corresponding sulfoxide. By modifying the oxidation conditions, it is also possible to prepare the corresponding sulfone.

These sulfides, sulfoxides and sulfones may then be converted in order to obtain the desired methacrylates or acrylates.

These monomeric compounds may be used as first monomer to prepare copolymers comprising them.

In particular, the monomeric compounds with an optical effect according to the invention may be used to prepare homopolymers or copolymers comprising only monomeric compounds with an optical effect, which may then each be present, for example, in a proportion of from 0.5% to 99.5% by weight, especially 5% to 95% by weight or even 10% to 90% by weight, and better still each in a proportion of 30% to 70% by weight relative to the total weight of the polymer. This may especially make it possible to prepare polymers with a wide range of optical effects (especially color, optical brightening or the like).

Statistical, alternating or grafted copolymers, comprising said monomeric compounds with an optical effect according to the invention and additional comonomers as defined below, may also be prepared.

Block copolymers, for example diblock or triblock copolymers, comprising said monomeric compounds with an optical effect according to the invention and additional comonomers as defined below, may also be prepared. The monomeric compounds according to the invention may form all or part of a block, or even several blocks. Block copolymers of the type A-B, ABA, BAB or ABC in which A is a block comprising the monomeric compound(s) according to the invention may thus be prepared, optionally as a mixture with additional comonomers, B and C being different blocks, comprising additional comonomers, alone or as a mixture, and identical to or different than the comonomers present in the block A.

The copolymers comprising the monomeric compounds according to the invention may also be of the gradient type.

In these copolymers, the monomeric compounds with an optical effect may be present in an amount of from 0.01% to 70% by weight relative to the weight of the final polymer, especially in an amount of from 0.1% to 50% by weight, in particular from 0.5% to 30% by weight or even from 1% to 20% by weight and better still from 2% to 10% by weight, the additional comonomers, alone or as a mixture, representing the remainder to 100% by weight.

The copolymers according to the invention may comprise, in addition to the monomeric compound(s) with an optical effect, at least one additional comonomer that is hydrophilic, or a mixture of such comonomers.

These hydrophilic comonomers may be present in a proportion of from 1% to 99.99% by weight, especially 2-70% by weight, better still 5-50% by weight or even 10-30% by weight, relative to the total weight of the copolymer.

In the present description, the term “hydrophilic monomer” will denote, without preference, monomers whose homopolymers are soluble or dispersible in water, or of which one ionic form is soluble or dispersible in water.

A homopolymer is said to be water-soluble if it forms a clear solution when it is in solution at 5% by weight in water, at 25° C.

A homopolymer is said to be water-dispersible if, at 5% by weight in water, at 25° C., it forms a stable suspension of fine, generally spherical particles. The mean size of the particles constituting said dispersion is less than 1 μm and more generally ranges between 5 and 400 nm and preferably from 10 to 250 nm. These particle sizes are measured by light scattering.

A monomer will be said to be “hydrophobic” if it is not hydrophilic.

Preferably, the additional hydrophilic comonomer(s) has a Tg of greater than or equal to 20° C. and especially greater than or equal to 50° C., but may optionally have a Tg of less than or equal to 20° C.

The copolymers according to the invention may comprise at least one additional hydrophobic comonomer, or a mixture of such comonomers.

These additional hydrophobic comonomers may be present in a proportion of from 1% to 99.99% by weight, especially 30-98% by weight, better still 50-95% by weight or even 70-90% by weight relative to the total weight of the copolymer.

Preferably, the hydrophobic comonomer has a Tg of greater than or equal to 20° C. and especially greater than or equal to 30° C., but may optionally have a Tg of less than or equal to 20° C.

In the present invention, the Tg (or glass transition temperature) is measured according to ASTM standard D3418-97, by differential thermal analysis (DSC “Differential Scanning Calorimetry”) on a calorimeter, over a temperature range of between −100° C. and +150° C. at a heating rate of 10° C./minute in 150 μl aluminum crucibles.

In general, as additional comonomer that may be copolymerized with at least one monomeric compound of formula (I), mention may be made, alone or as a mixture, of the following monomers:
(i) ethylenic hydrocarbons containing from 2 to 10 carbons, such as ethylene, isoprene or butadiene;
(ii) the (meth)acrylates of formula:
   CH₂═CHCOOR₃ or 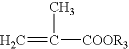
   in which R₃ represents:
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F), and groups Si(R₄R₅), in which R₄ and R₅, which may be identical or different, represent a C₁ to C₆ alkyl group or a phenyl group;
   R₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
   said cycloalkyl, aryl, aralkyl, heterocyclic or heterocycloalkyl groups possibly being optionally substituted with one or more substitutents chosen from hydroxyl groups, halogen atoms and linear or branched C₁₋₄ alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F), and groups Si (R₄R₅), in which R₄ and R₅, which may be identical or different, represent a C₁ to C₆ alkyl group or a phenyl group,
R₃ may also be a group —(C₂H₄O)ₘ—R″, with m=5 to 150 and R″═H or C₁ to C₃₀ alkyl, for example —POE-methyl or —POE-behenyl;
(iii) the (meth)acrylamides of formula: 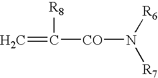
   in which R₈ denotes H or methyl; and R₇ and R₆, which may be identical or different, represent:
a hydrogen atom; or
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F), and groups Si(R₄R₅), in which R₄ and R₅, which may be identical or different, represent a C₁ to C₆ alkyl group or a phenyl group;
   R₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group, such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
   said cycloalkyl, aryl, aralkyl, heterocyclic or heterocycloalkyl groups possibly being optionally substituted with one or more substitutents chosen from hydroxyl groups, halogen atoms and linear or branched C₁-C₄ alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F) and groups Si(R₄R₅), in which R₄ and R₅, which may be identical or different, represent a C₁ to C₆ alkyl group or a phenyl group.

Examples of (meth)acrylamide monomers are (meth)acrylamide, N-ethyl(meth)acrylamide, N-butyl-acrylamide, N-t-butylacrylamide, N-isopropylacrylamide, N,N-dimethyl(meth)acrylamide, N,N-dibutylacrylamide, N-octylacrylamide, N-dodecylacrylamide, undecyl-acrylamide and N(2-hydroxypropylmethacrylamide).
(iv) the vinyl compounds of formulae:
   CH₂═CH—R₉, CH₂═CH—CH₂—R₉ or CH₂═C(CH₃)—CH₂—R₉
   in which R₉ is a hydroxyl group, halogen (Cl or F), NH₂, OR₁₀ in which R₁₀ represents a phenyl group or a C₁ to C₁₂ alkyl group (the monomer is a vinyl or allylic ether); acetamide(NHCOCH₃); a group OCOR₁₁ in which R₁₁ represents a linear or branched alkyl group of 2 to 12 carbons (the monomer is a vinyl or allylic ester); or a group chosen from:
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F) and groups Si(R₄R₅), in which R₄ and R₅, which may be identical or different, represent a C₁ to C₆ alkyl group or a phenyl group;
a C₃ to C₁₂ cycloalkyl group such as isobornyl or cyclohexane,
a C₃ to C₂₀ aryl group such as phenyl,
a C₄ to C₃₀ aralkyl group (C₁ to C₈ alkyl group) such as 2-phenylethyl; benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
   said cycloalkyl, aryl, aralkyl, heterocyclic or heterocycloalkyl groups possibly being optionally substituted with one or more substitutents chosen from hydroxyl groups, halogen atoms and linear or branched 1 to 4 C alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F) and groups Si(R₄R₅) in which R₁ and R₂, which may be identical or different, represent a C₁ to C₆ alkyl group, or a phenyl group.

Examples of vinyl monomers are vinyl cyclohexane and styrene.

Examples of vinyl esters are vinyl acetate, vinyl propionate, vinyl butyrate, vinyl ethylhexanoate, vinyl neononanoate and vinyl neododecanoate.

Among the vinyl ethers that may be mentioned are methyl vinyl ether, ethyl vinyl ether and isobutyl vinyl ether.
(v) (meth)acrylic, (meth)acrylamide or vinyl monomers containing a fluoro or perfluoro group, such as ethylperfluorooctyl or 2-ethylperfluorohexyl (meth)acrylate;
(vi) silicone-based (meth)acrylic, (meth)acrylamide or vinyl monomers, such as methacryloxypropyltris(trimethylsiloxy)silane or acryloxypropylpolydimethylsiloxane;
(vii) ethylenically unsaturated monomers comprising at least one carboxylic, phosphoric or sulfonic acid, or anhydride, function, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;
(viii) ethylenically unsaturated monomers comprising at least one tertiary amine function, for instance 2-vinylpyridine, 4-vinylpyridine, dimethylaminoethyl methacrylate, diethylaminoethyl methacrylate and dimethylaminopropylmethacrylamide, and the salts thereof.

The salts may be formed by neutralization of the anionic groups with a mineral base, such as LiOH, NaOH, KOH, Ca(OH)₂, NH₄OH or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of amino-2-methyl-2-propanol, triethanolamine and dimethylamino-2-propanol. Mention may also be made of lysine or 3-(dimethylamino)propylamine.

Mention may also be made of the salts of mineral acids, such as sulfuric acid, hydrochloric acid, hydrobromic acid, hydriodic acid, phosphoric acid or boric acid. Mention may also be made of the salts of organic acids, which may comprise one or more carboxylic, sulfonic or phosphonic acid groups. They may be linear, branched, or cyclic aliphatic acids, or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

It is obviously possible to use several of the additional comonomers mentioned above.

The additional comonomer(s) may be present in an amount of from 30% to 99.99% by weight, especially in an amount of from 50% to 99.9% by weight, in particular from 70% to 99.5% by weight, or even from 80% to 99% by weight, and better still from 90% to 98% by weight, relative to the weight of the final polymer.

The additional comonomers are more particularly chosen, alone or as a mixture, from C₁-C₁₈ alkyl or C₃-C₁₂ cycloalkyl(meth)acrylates, and especially from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate.

Mention may also be made of acrylic acid, methacrylic acid, methacryloxypropyltris(trimethylsiloxy) silane, acryloxypropyltris(trimethylsiloxy)silane, acryloxy-propylpolydimethylsiloxane and methacryloxypropylpoly-dimethylsiloxane.

Said polymers may be prepared according to the methods known to those skilled in the art, especially by radical polymerization; controlled radical polymerization, for example with xanthans, dithiocarbamates or dithio esters; by polymerization using precursors of nitroxide type; by atom transfer radical polymerization (ATRP); by group transfer polymerization.

The polymerization may conventionally be performed in the presence of a polymerization initiator, which may be a radical initiator, and especially which may be chosen from organic peroxide compounds such as dilauroyl peroxide, dibenzoyl peroxide, tert-butyl peroxy-2-ethylhexanoate; or from diazo compounds such as azobisisobutyronitrile or azobisdimethylvalero-nitrile. The reaction may also be initiated using photoinitiators or via radiation of UV type, with neutrons or with plasma.

The monomeric compounds with an optical effect, and also the homopolymers or copolymers comprising them, preferably have an absorption wavelength of between 200 and 500 nm, especially between 220 and 450 nm or even between 240 and 440 nm.

They preferably have an emission wavelength of between 350 and 700 nm, especially between 390 and 650 nm, or even between 400 and 600 nm.

The weight-average molecular mass (Mw) of the copolymers according to the invention is preferably between 5000 and 600 000 g/mol, especially between 10 000 and 300 000 g/mol and better still between 20 000 and 150 000 g/mol.

The weight-average (Mw) and number-average (Mn) molecular masses are determined by gel permeation liquid chromatography (GPC), eluting with THF, on a calibration curve established with linear polystyrene standards, using a refractometric detector.

The polymers according to the invention, whether they are homopolymers or copolymers, may be present, alone or as a mixture, in the compositions according to the invention in an amount of from 0.01% to 60% by weight, preferably 0.1% to 50% by weight, especially 1% to 25% by weight, or even 3% to 15% by weight and better still 5% to 12% by weight, relative to the total weight of the composition.

They may be present in the composition in dissolved form, for example in water, in an oil or in an organic solvent, or alternatively in the form of an aqueous or organic dispersion.

Advantageously, the polymers according to the invention are soluble or dispersible in at least one of the phases of the composition comprising them.

The cosmetic or pharmaceutical compositions according to the invention comprise, besides said polymers, a physiologically acceptable medium, especially a cosmetically, dermatologically or pharmaceutically acceptable medium, i.e. a medium that is compatible with keratin materials such as facial or bodily skin, the hair, the eyelashes, the eyebrows and the nails.

The composition may thus comprise a hydrophilic medium comprising water or a mixture of water and hydrophilic organic solvent(s), for instance alcohols and especially linear or branched lower monoalcohols containing from 2 to 5 carbon atoms, for instance ethanol, isopropanol or n-propanol, and polyols, for instance glycerol, diglycerol, propylene glycol, sorbitol or pentylene glycol, and polyethylene glycols, or alternatively hydrophilic C₂ ethers and C₂-C₄ aldehydes.

The water or the mixture of water and hydrophilic organic solvents may be present in the composition according to the invention in a content ranging from 0.1% to 99% by weight and preferably from 10% to 80% by weight relative to the total weight of the composition.

The composition may also be anhydrous.

The composition may also comprise a fatty phase which may comprise fatty substances that are liquid at room temperature (in general 25° C.) and/or fatty substances that are solid at room temperature, such as waxes, pasty fatty substances and gums, and mixtures thereof. These fatty substances may be of animal, plant, mineral or synthetic origin. This fatty phase may also contain lipophilic organic solvents.

As fatty substances that are liquid at room temperature, often referred to as oils, which may be used in the invention, mention may be made of: hydrocarbon-based oils of animal origin such as perhydrosqualene; hydrocarbon-based plant oils such as liquid triglycerides of fatty acids of 4 to 10 carbon atoms, for instance heptanoic or octanoic acid triglycerides, or alternatively sunflower oil, maize oil, soybean oil, grapeseed oil, sesame seed oil, apricot oil, macadamia oil, castor oil, avocado oil, caprylic/capric acid triglycerides, jojoba oil, shea butter, linear or branched hydrocarbons of mineral or synthetic origin, such as liquid paraffin and derivatives thereof, petroleum jelly, polydecenes, hydrogenated polyisobutene such as parleam; synthetic esters and ethers, especially of fatty acids, for instance purcellin oil, isopropyl myristate, 2-ethyl-hexyl palmitate, 2-octyldodecyl stearate, 2-octyl-dodecyl erucate, isostearyl isostearate; hydroxylated esters, for instance isostearyl lactate, octyl hydroxy-stearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and pentaerythritol esters; fatty alcohols containing from 12 to 26 carbon atoms, for instance octyldodecanol, 2-butyloctanol, 2-hexyl-decanol, 2-undecylpentadecanol and oleyl alcohol; partially hydrocarbon-based fluoro oils and/or partially silicone-based fluoro oils; silicone oils, for instance volatile or non-volatile, linear or cyclic polymethylsiloxanes (PDMSs), which are liquid or pasty at room temperature, for instance cyclomethicones, dimethicones, optionally comprising a phenyl group, for instance phenyl trimethicones, phenyltrimethylsiloxy-diphenylsiloxanes, diphenylmethyldimethyltrisiloxanes, diphenyl dimethicones, phenyl dimethicones and polymethylphenylsiloxanes; mixtures thereof.

These oils may be present in a content ranging from 0.01% to 90% and better still from 0.1% to 85% by weight relative to the total weight of the composition.

The composition according to the invention may also comprise one or more physiologically acceptable organic solvents.

These solvents may be generally present in a content ranging from 0.1% to 90%, preferably from 0.5% to 85%, more preferably from 10% to 80% and better still from 30% to 50% by weight, relative to the total weight of the composition.

Mention may be made especially, besides the hydrophilic organic solvents mentioned above, of ketones that are liquid at room temperature such as methyl ethyl ketone, methyl isobutyl ketone, diisobutyl ketone, isophorone, cyclohexanone and acetone; propylene glycol ethers that are liquid at room temperature, such as propylene glycol monomethyl ether, propylene glycol monomethyl ether acetate, and dipropylene glycol mono-n-butyl ether; short-chain esters (containing from 3 to 8 carbon atoms in total), such as ethyl acetate, methyl acetate, propyl acetate, n-butyl acetate and isopentyl acetate; ethers that are liquid at 25° C., such as diethyl ether, dimethyl ether or dichlorodiethyl ether; alkanes that are liquid at 25° C., such as decane, heptane, dodecane, isododecane and cyclohexane; aromatic cyclic compounds that are liquid at 25° C., such as toluene and xylene; aldehydes that are liquid at 25° C., such as benzaldehyde and acetaldehyde, and mixtures thereof.

For the purposes of the present invention, the term “wax” means a lipophilic compound that is solid at room temperature (25° C.), which undergoes a reversible solid/liquid change of state, and which has a melting point of greater than or equal to 25° C., which may be up to 120° C. By bringing the wax to the liquid state (melting), it is possible to make it miscible with the oils possibly present and to form a microscopically homogeneous mixture, but, on returning the temperature of the mixture to room temperature, recrystallization of the wax is obtained in the oils of the mixture. The melting point of the wax may be measured using a differential scanning calorimeter (DSC), for example the calorimeter sold under the name DSC 30 by the company Mettler.

The waxes may be hydrocarbon-based waxes, fluoro waxes and/or silicone waxes and may be of plant, mineral, animal and/or synthetic origin. In particular, the waxes have a melting point of greater than 30° C. and better still greater than 45° C. As waxes that may be used in the composition of the invention, mention may be made of beeswax, carnauba wax or candellila wax, paraffin, microcrystalline waxes, ceresin or ozokerite, synthetic waxes, for instance polyethylene waxes or Fischer-Tropsch waxes, and silicone waxes, for instance alkyl or alkoxy dimethicones containing from 16 to 45 carbon atoms.

The gums are generally polydimethylsiloxanes (PDMSs) of high molecular weight or cellulose gums or polysaccharides, and the pasty substances are generally hydrocarbon-based compounds, for instance lanolins and derivatives thereof, or PDMSs.

The nature and amount of the solid substances depend on the desired mechanical properties and textures. As a guide, the composition may contain from 0.1% to 50% by weight and better still from 1% to 30% by weight of waxes relative to the total weight of the composition.

The composition according to the invention may also comprise, in a particulate phase, pigments and/or nacres and/or fillers usually used in cosmetic compositions.

The composition may also comprise other dyestuffs chosen from water-soluble dyes and/or liposoluble dyes that are well known to those skilled in the art.

The term “pigments” should be understood as meaning white or colored, mineral or organic particles of any shape, which are insoluble in the physiological medium and which are intended to color the composition.

The term “fillers” should be understood as meaning colorless or white, mineral or synthetic, lamellar or non-lamellar particles intended to give body or rigidity to the composition, and/or softness, a matt effect and uniformity to the makeup result.

The term “nacres” should be understood as meaning iridescent particles of any form, produced especially by certain molluscs in their shell, or else synthesized.

The pigments may be present in the composition in a proportion of from 0.01% to 25% and preferably in a proportion of from 3% to 10% by weight of the final composition. They may be white or colored, and mineral or organic. Mention may be made of titanium oxide, zirconium oxide or cerium oxide, and also zinc oxide, iron oxide or chromium oxide, ferric blue, chromium hydrate, carbon black, ultramarines (aluminosilicate polysulfides), manganese pyrophosphate and certain metallic powders such as silver or aluminum powder. Mention may also be made of the D&C pigments and lakes commonly used to give the lips and the skin a makeup effect, which are calcium, barium, aluminum, strontium or zirconium salts.

The nacres may be present in the composition in a proportion of from 0.01% to 20% by weight and preferably in a proportion of about from 3% to 10% by weight. Among the nacres that may be envisaged, mention may be made of natural mother-of-pearl, mica coated with titanium oxide, with iron oxide, with natural pigment or with bismuth oxychloride, and also colored titanium mica.

Among the liposoluble or water-soluble dyes that may be present in the composition, alone or as a mixture, in a proportion of from 0.001% to 15% by weight, preferably 0.01% to 5% by weight and especially from 0.1% to 2% by weight, relative to the total weight of the composition, mention may be made of the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll, methylene blue, cochineal carmine, halo-acid dyes, azo dyes, anthraquinone dyes, copper sulfate, iron sulfate, Sudan brown, Sudan red and annatto, and also beetroot juice and carotene.

The composition according to the invention may also comprise one or more fillers, especially in a content ranging from 0.01% to 50% by weight and preferably ranging from 0.02% to 30% by weight, relative to the total weight of the composition. The fillers may be mineral or organic in any form, platelet-shaped, spherical or oblong. Mention may be made of talc, mica, silica, kaolin, polyamide (Nylon®) powder, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The composition may also comprise an additional polymer such as a film-forming polymer. According to the present invention, the term “film-forming polymer” means a polymer capable, by itself or in the presence of an auxiliary film-forming agent, of forming a continuous film that adheres to a support and especially to keratin materials. Among the film-forming polymers that may be used in the composition of the present invention, mention may be made of synthetic polymers, of free-radical type or of polycondensate type, polymers of natural origin and mixtures thereof, in particular acrylic polymers, polyurethanes, polyesters, polyamides, polyureas and cellulose-based polymers, for instance nitrocellulose.

The composition according to the invention may also comprise ingredients commonly used in cosmetics, such as vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants and ceramides, or mixtures thereof. Needless to say, a person skilled in the art will take care to select this or these optional additional compound(s), and/or the amount thereof, such that the advantageous properties of the composition according to the invention are not, or are not substantially, adversely affected by the envisaged addition.

The composition according to the invention may be in the form of a suspension, a dispersion, especially of oil in water by means of vesicles; an optionally thickened or even gelled oily solution; an oil-in-water, water-in-oil or multiple emulsion; a gel or a mousse; an oily or emulsified gel; a dispersion of vesicles, especially of lipid vesicles; a two-phase or multiphase lotion; a spray; a loose, compact or cast powder; an anhydrous pastep. This composition may have the appearance of a lotion, a cream, a pomade, a soft pastep, an ointment, a cast or molded solid especially as a stick or in a dish, or alternatively a compacted solid.

A person skilled in the art will be able to choose the appropriate galenical form, and also the method for preparing it, on the basis of his general knowledge, taking into account firstly the nature of the constituents used, especially their solubility in the support, and secondly the intended application of the composition.

The cosmetic composition according to the invention may be in the form of a care and/or makeup product for bodily or facial skin, the lips, the nails, the eyelashes, the eyebrows and/or the hair, an antisun or self-tanning product, or a hair product for caring for, treating, shaping, making up or dyeing the hair.

It may also be in the form of a makeup composition, especially a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer).

It may also be in the form of a composition for protecting or caring for the skin of the face, the neck, the hands or the body, especially an antiwrinkle composition or a moisturizing or treating composition; an antisun composition or artificial tanning composition.

It may also be in the form of a hair product, especially for dyeing, holding the hairstyle, shaping the hair, caring for, treating or cleansing the hair, such as shampoos, hairsetting gels or lotions, blow-drying lotions, and fixing and styling compositions such as lacquers or sprays.

A subject of the invention is also a cosmetic process for making up or caring for keratin materials, especially bodily or facial skin, the lips, the nails, the eyelashes, the eyebrows and/or the hair, comprising the application to said materials of a cosmetic composition as defined above.

The invention is illustrated in greater detail in the examples that follow.

Method for Measuring the Wavelength (Emission and Absorption

The wavelength measurement is performed using a Varian Cary Eclipse fluorimeter.

Unless otherwise mentioned, this measurement is performed in the following manner: 20 mg of product are placed in a 50 ml cylinder. To dissolve the product, said cylinder is filled to 50 ml with a suitable solvent, for example dichloromethane (DCM), chloroform or dimethyl sulfoxide (DMSO). The resulting solution is mixed and 250 microliters are taken and placed in a 50 ml cylinder, and the volume is then made up to 50 ml again with the solvent.

The whole is mixed and a sample of the solution is taken and placed in a closed quartz cell 10 mm thick, which is then placed in the measuring chamber.

### EXAMPLE 1

1. First Step

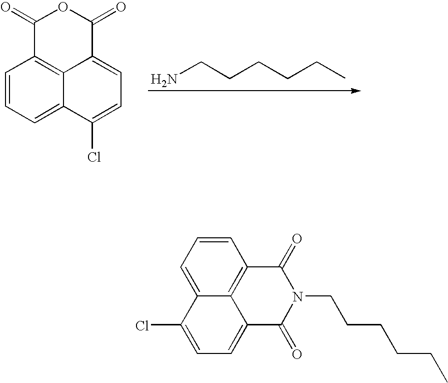

26.9 g (0.116 mol) of 4-chloro-1,8-naphthalic anhydride are placed in a 2 liter round-bottomed flask, under an inert atmosphere (nitrogen), and 450 ml of toluene are then added. The mixture is stirred at 500 rpm for a few minutes, and 14.0 g (0.139 mol) of 1-hexylamine predissolved in 100 ml of toluene are then added dropwise. The mixture is heated to reflux and a further 50 ml of toluene are then added. Refluxing is continued for 24 hours. The resulting reaction mixture is then allowed to cool to room temperature. The product is concentrated under reduced pressure. The organic phase is recovered and recrystallized from ethanol. 29.0 g of pale yellow crystals are obtained (79.4% yield).

Characterization

¹H NMR (CDCl₃, 400 MHz) δ: 8.67-8.65 (2H), 8.51-8.49 (1H), 7.87-7.85 (1H), 7.83-7.81 (1H), 4.18-4.14 (2H), 1.74-1.70 (2H), 1.44-1.32 (6H), 0.90-0.87 (3H).
2. Second Step

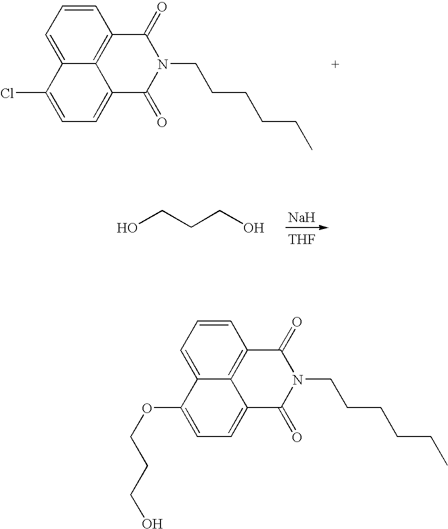

1.5 g (64 mmol) of sodium hydride (NaH) are placed in a round-bottomed flask under an inert atmosphere of argon; 300 ml of THF (tetrahydrofuran) are added and the reaction mixture is cooled to 0° C. 2.4 g (32 mmol) of 1,3-propanediol premixed with 100 ml of THF are added, and the mixture is stirred vigorously. The mixture is heated at 60° C. for 20 minutes and then cooled to room temperature (25° C.). A mixture consisting of 10 g (32 mmol) of naphthalimide in 150 ml of THF is then added. The mixture is refluxed for 3 hours, 10 ml of ethanol are then added, this mixture is heated for 15 minutes, and the solvent is evaporated off under reduced pressure to give a greenish residue, which is washed with ethyl acetate and water. 8.6 g of a pale yellow powder are thus obtained (75.5% yield).

Characterization

¹H NMR (CDCl₃, 400 MHz) δ: 8.61-8.59 (2H), 8.55-8.53 (1H), 7.72-7.68 (1H), 7.08-7.06 (1H), 4.45-4.42 (2H), 4.17-4.13 (2H), 4.00-3.97 (2H), 2.28-2.22 (2H), 1.76-1.70 (2H), 1.44-1.32 (6H), 0.90-0.87 (3H).
3. Third Step

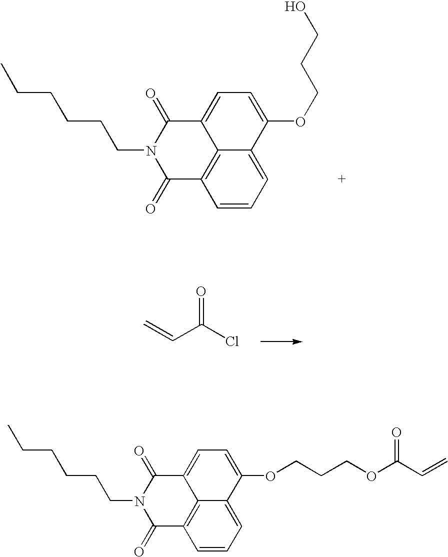

8.0 g (22.5 mmol) of 4-(oxy-3-propanol)-N-hexyl-naphthalimide are placed in a three-necked round-bottomed flask equipped with a condenser and placed under an inert atmosphere of argon. 150 ml of dichloromethane are added and the solution is stirred until homogeneous. 4.6 g (45.0 mmol) of triethanolamine are then added, followed by addition of 4.1 g (45.0 mmol) of acryloyl chloride, with stirring at 0° C. The reaction progress is monitored by TLC (thin-layer chromatography), and when there are no more starting compounds remaining, 50 ml of water are added. The reaction solution is then washed with saturated NaCl solution and then dried over sodium sulfate. The solvents are evaporated off under reduced pressure to give 9.2 g of a pale yellow powder (100% yield).

Characterization

¹H NMR (CDCl₃, 400 MHz) δ: 8.59-8.57 (1H), 8.54-8.52 (1H), 8.51-8.50 (1H), 7.70-7.66 (1H), 7.03-7.01 (1H), 6.44-6.40 (1H), 6.17-6.10 (1H), 5.86-5.83 (1H), 4.49-4.46 (2H), 4.39-4.36 (2H), 4.16-4.12 (2H), 2.38-2.35 (2H), 1.71-1.69 (2H), 1.44-1.31 (6H), 0.89-0.86 (3H).
absorption wavelength: 2 absorption peaks are observed at 248 nm and 365 nm.
emission wavelength λₘₐₓ emission: about 430 nm (violet-blue)
   (solvent: chloroform)

### EXAMPLE 2

The compounds below are prepared in a manner similar to that of example 1: 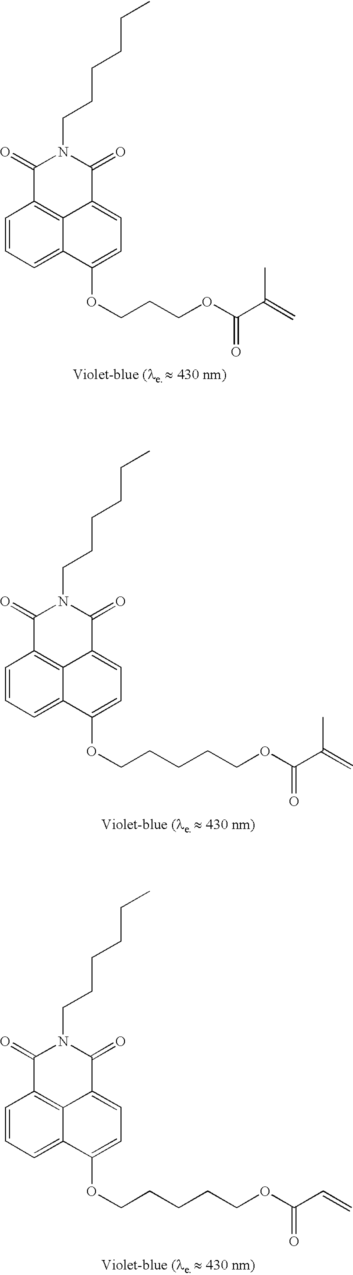 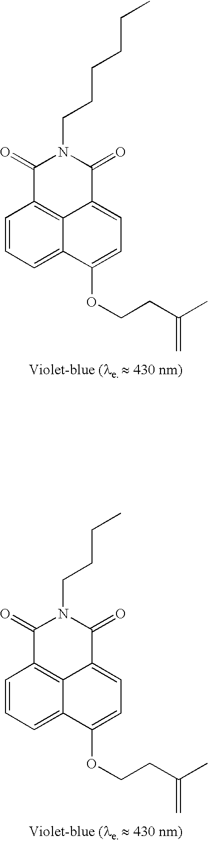
(solvent for the wavelength measurement: DCM)

### EXAMPLE 3

1. First Step

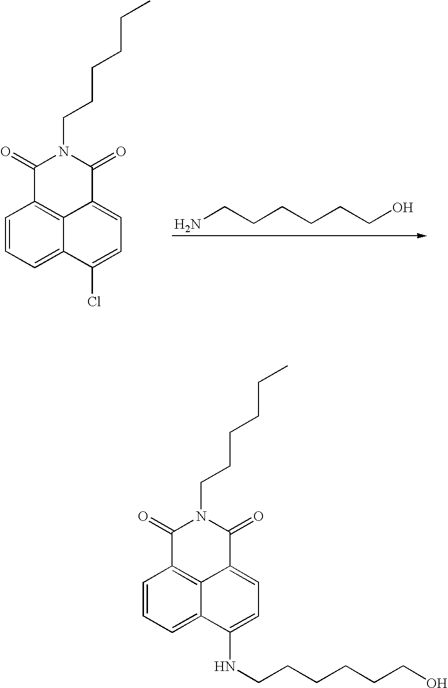

2.71 g (10.85 mmol) of CuSO₄.5H₂O and 10.07 g (31.9 mmol) of 4-chloro-N-hexyl-1,8-naphthalimide are mixed together in 170 ml of ethanol in a round-bottomed flask, under argon, and the reaction medium is then brought to reflux.

7.7 g (65.7 mmol) of amino-6-hexanol are added dropwise to the reaction medium, and stirring is then maintained for 16 hours. The solution is then cooled to 25° C. and the product is precipitated by addition of 200 ml of water and then filtered through a sinter funnel. A yellow-green solid product is obtained, which is taken up in 100 ml of dichloromethane. The aqueous phase is washed with water and then evaporated to give an oil, which is filtered through silica (0.043-0.060 microns, eluting with dichloromethane and then with acetone).

1.6 g of a yellow oil are finally obtained (12.6% yield).

Characterization

¹H NMR (CDCl₃, 400 MHz) δ ppm: 0.87 (t, 3H, CH₃); 1.50 (m, 8H, CH₂); 1.81 (m, 2H, NCH₂CH₂); 3.40 (t, 2H, CH₂OH); 3.68 (t, 2H, NHCH₂); 4.14 (t, 2H, NCH₂); 5.26 (s, 1H, NH); 6.71 (d, 1H, ArH); 7.60 (t, 1H, ArH); 8.06 (d, 1H, ArH); 8.43 (d, 1H, ArH); 8.57 (d, 1H, ArH).
2. Second Step

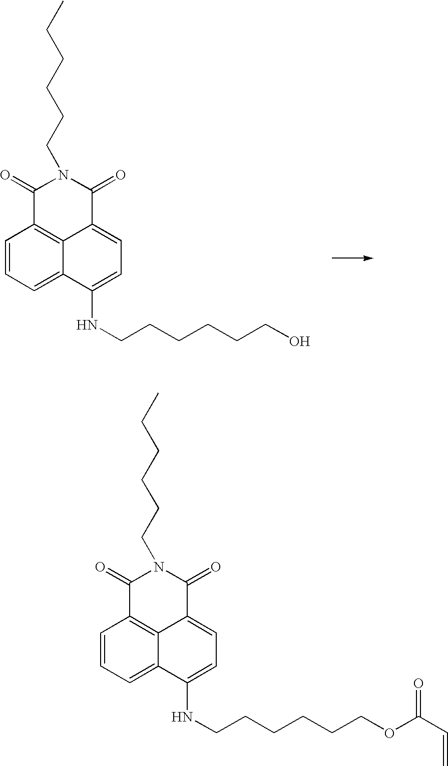

1.5 g (3.9 mmol) of 4-(amino-6-hexanol)-N-hexyl-naphthalimide are placed in a three-necked round-bottomed flask equipped with a condenser and placed under an inert atmosphere of argon. 50 ml of dichloromethane are added and the solution is stirred until homogeneous. 0.8 g (7.9 mmol) of triethanolamine is then added, followed by 0.7 g (7.9 mmol) of acryloyl chloride, with stirring at 0° C. The temperature is then allowed to rise to 25° C. The reaction progress is monitored by TLC and, when it is observed that there are no remaining starting products, 10 ml of water are added. The reaction solution is then washed with saturated NaCl solution and then dried over sodium sulfate. The solvents are evaporated off under reduced pressure to give 1.7 g (99% yield) of an orange powder.

Characterization

¹H NMR (CDCl₃, 400 MHz) δ ppm: 0.87 (t, 3H, CH₃); 1.50 (m, 8H, CH₂); 1.81 (m, 2H, NCH₂CH₂); 3.68 (t, 2H, NHCH₂); 4.17 (m, 4H, NCH₂; CH₂O); 5.26 (s, 1H, NH); 5.77 (d, 1H C═CH); 6.10 (m, 1H C═CH); 6.39 (d, 1H, CH═C); 6.70 (d, 1H, ArH); 7.60 (t, 1H, ArH); 8.06 (d, 1H, ArH); 8.43 (d, 1H, ArH); 8.57 (d, 1H, ArH).
emission wavelength λₑₘᵢₛₛᵢₒₙ: about 501 nm
   (solvent: DCM)

### EXAMPLE 4

1. First Step

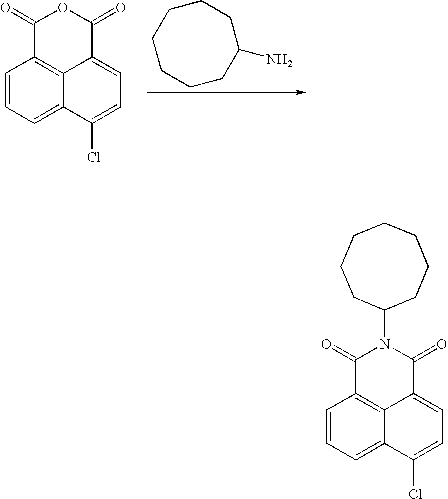

20.0 g (0.086 mol) of 4-chloro-1,8-naphthalic anhydride are placed in a 2 liter round-bottomed flask, under an inert atmosphere (argon), followed by addition of 250 ml of toluene. The mixture is heated at 100° C. and stirred until the solution becomes homogeneous. It is then stirred (500 rpm) and 12.0 g (0.095 mol) of cyclo-octylamine predissolved in 50 ml of toluene are then added dropwise. The mixture is heated to reflux. Refluxing is continued for 5 hours. The resulting reaction mixture is then allowed to cool to room temperature. The solid phase is recovered by filtration, washed with water and dried. 23.9 g of a pale yellow powder are obtained (81.2% yield).

Characterization

¹H NMR (CDCl₃, 400 MHz) δ: 8.63-8.61 (1H), 8.57-8.55 (1H), 8.47-8.45 (1H), 7.85-7.79 (2H), 5.32-5.27 (1H), 2.52-2.44 (2H), 1.86-1.57 (12H).
2. Second Step

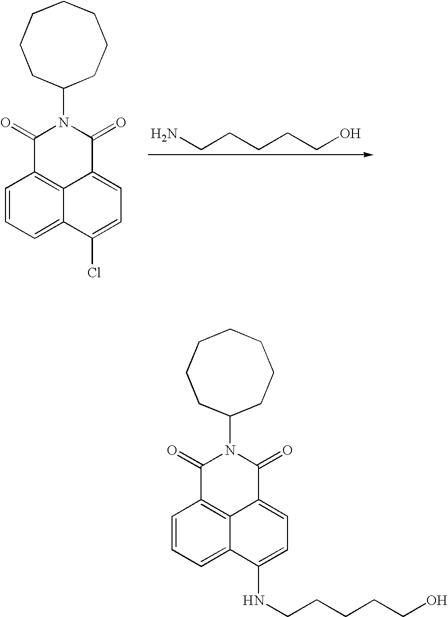

20.0 g (0.059 mol) of N-cyclooctyl-4-chloro-1,8-naphthalimide are placed in a three-necked round-bottomed flask equipped with a condenser and placed under an inert atmosphere of argon. 18.1 g (0.176 mol) of 5-pentanolamine are then added and the mixture is heated to 140° C. so as to obtain a homogeneous solution. This solution is left to react at 140° C. for 3 hours, and is then allowed to cool to room temperature. The reaction solution is mixed with 125 ml of dichloromethane and then washed with sodium bicarbonate. The product is precipitated from dichloromethane and the precipitate is filtered off, washed with water and dried.

24.1 g of yellow crystals are obtained.

Characterization

¹H NMR (CDCl₃, 400 MHz) δ: 8.55-8.42 (2H), 8.07-8.04 (1H), 7.61-7.57 (1H), 6.72-6.70 (1H), 5.35-5.30 (1H), 3.74-3.71 (2H), 3.43-3.40 (2H), 2.54-2.46 (2H)—, 1.90-1.54 (19H).
3. Third Step

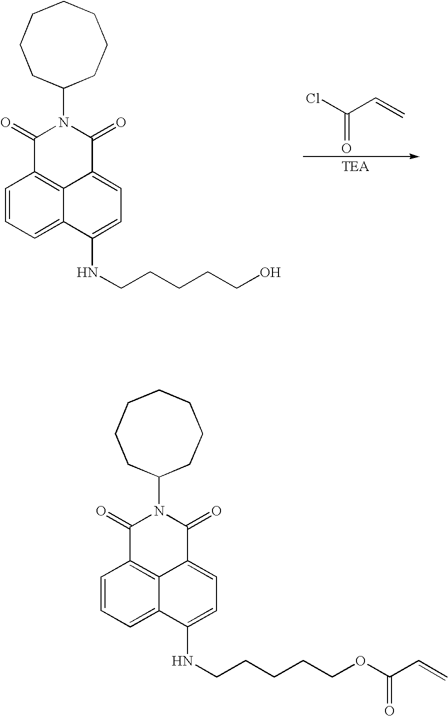

15.0 g (0.037 mol) of N-cyclooctyl-4-(amino-5-pentanol)-1,8-naphthalimide are placed in a 1 liter round-bottomed flask, under an inert atmosphere (argon), and 175 ml of dichloromethane are then added. The mixture is stirred until a homogeneous solution is obtained. 11.1 g (0.110 mol) of triethanolamine are then added. A mixture of 3.7 g (0.040 mol) of acryloyl chloride in 10 ml of THF is then added dropwise with stirring (500 rpm) at 38° C. The mixture is left to react for 2 hours and the solvent is then evaporated off under reduced pressure. Dichloromethane is added to the organic phase, which is then washed with water and sodium bicarbonate, and then again with water. The organic phase is dried over sodium sulfate and filtered. The organic phase is evaporated and 16.1 g of product are recovered (yield: 94.8%).

Characterization

¹H NMR (CDCl₃, 400 MHz) δ: 8.54-8.52 (1H), 8.43-8.41 (1H), 8.11-8.08 (1H), 6.71-6.69 (2H), 6.42-6.38 (1H), 6.15-6.08 (1H), 5.84-5.81 (1H), 5.31-5.29 (1H), 4.23-4.20 (2H), 3.75-3.72 (1H), 3.44-3.39 (2H), 2.53-2.45 (2H), 1.86-1.56 (18H).
absorption wavelength λ_{absorption}: 370 nm
emission wavelength λₘₐₓ emission: 505 nm (orange)
   (solvent: DCM)

### EXAMPLE 5

A statistical copolymer comprising a monomer according to the invention is prepared.

20 g of isododecane are placed in a reactor, under argon, equipped with a condenser and with stirring, followed by 39.0 g of isobornyl acrylate and 10.0 g of ethylhexyl acrylate. The mixture is stirred and a mixture consisting of 1.0 g of N-hexyl-1,8-naphthalimide-4-oxypropane acrylate (monomer of example 1) in 20.0 g of toluene is then added.

0.5 g of Trigonox 21S (t-butyl peroxy-2-ethylhexanoate) is added and the reaction mixture is then heated to 90° C.; the stirring and heating are continued for 6 hours and the mixture is then cooled to room temperature. The resulting polymer is purified by precipitation.

A statistical polymer comprising (weight %): 78% isobornyl acrylate, 20% ethylhexyl acrylate and 2% monomer according to the invention is obtained.
molecular weight Mw=34 000
absorption wavelength λ_{absorption}: 250 nm and 366 nm
emission wavelength % max emission: 430 nm
   (solvent: DCM)

### EXAMPLE 6

A homopolymer is prepared from a monomer according to the invention

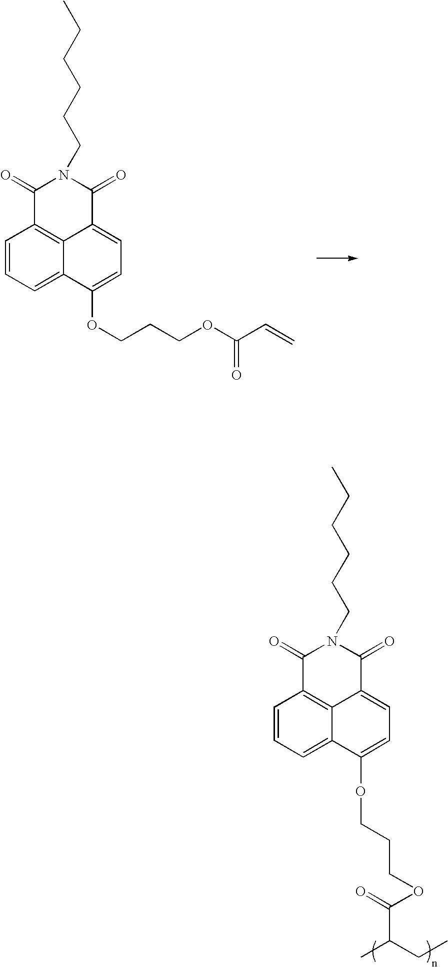

3.0 g (7.3 mmol) of monomer prepared in example 1 are dissolved in 8.5 ml of anhydrous toluene, at 60° C. in the presence of Trigonox 21S (180 μL). The mixture is heated to 90° C. and stirring is continued for 18 hours. Since the viscosity of the solution increases, the medium is diluted by adding 20 ml of toluene and is then precipitated dropwise from 500 ml of acetone cooled to −10° C. The polymer is then dried in an oven (50° C.) under vacuum.

2.3 g of polymer are obtained, i.e. a yield of 77%.

An emission wavelength λₘₐₓ emission of 458 nm is observed.

(solvent: DCM)

### EXAMPLE 7

A statistical copolymer comprising a monomer according to the invention is prepared. 20 g of isododecane are placed in a reactor, under argon, equipped with a condenser and a stirrer, followed by 38.25 g of isobornyl acrylate and 10.0 g of ethylhexyl acrylate. The mixture is stirred and a mixture consisting of 1.75 g of N-hexyl-1,8-naphthalimide-4-oxypropane acrylate (monomer of example 1) in 20.0 g of toluene is added.

0.5 g of Trigonox 21S (t-butyl peroxy-2-ethylhexanoate) is added and the reaction mixture is then heated to 90° C.; the stirring and heating are continued for 6 hours and the mixture is then cooled to room temperature. The resulting polymer is purified by precipitation.

A statistical polymer comprising (weight %): 76.5% isobornyl acrylate, 20% ethylhexyl acrylate and 3.5% monomer according to the invention is obtained.
molecular weight Mw=37 300

### EXAMPLE 8

A statistical copolymer comprising a monomer according to the invention is prepared. 20 g of isododecane are placed in a reactor, under argon, equipped with a condenser and a stirrer, followed by 39.75 g of isobornyl acrylate and 10.0 g of ethylhexyl acrylate. The mixture is stirred and a mixture consisting of 0.25 g of N-hexyl-1,8-naphthalimide-4-oxypropane acrylate (monomer of example 1) in 20.0 g of toluene is added.

0.5 g of Trigonox 21S (tert-butyl peroxy-2-ethyl-hexanoate) is added and the reaction mixture is then heated to 90° C.; the stirring and heating are continued for 6 hours and the mixture is then cooled to room temperature. The resulting polymer is purified by precipitation.

A statistical polymer comprising (weight %): 79.5% isobornyl acrylate, 20% ethylhexyl acrylate and 0.5% monomer according to the invention is obtained.
molecular weight Mw=35 200

### EXAMPLE 9

A statistical copolymer comprising a monomer according to the invention is prepared. 20 g of isododecane are placed in a reactor, under argon, equipped with a condenser and a stirrer, followed by 37.0 g of isobornyl acrylate and 10.0 g of ethylhexyl acrylate. The mixture is stirred and a mixture consisting of 3.0 g of N-hexyl-1,8-naphthalimide-4-oxypropane acrylate (monomer of example 1) in 20.0 g of toluene is added.

0.5 g of Trigonox 21S (tert-butyl peroxy-2-ethyl-hexanoate) is added and the reaction mixture is then heated to 90° C.; the stirring and heating are continued for 6 hours and the mixture is then cooled to room temperature. The resulting polymer is purified by precipitation.

A statistical polymer comprising (weight %): 74% isobornyl acrylate, 20% ethylhexyl acrylate and 6% monomer according to the invention is obtained.

### EXAMPLE 10

A statistical copolymer comprising a monomer according to the invention is prepared. 20 g of isododecane are placed in a reactor, under argon, equipped with a condenser and a stirrer, followed by 35.0 g of isobornyl acrylate and 10.0 g of ethylhexyl acrylate. The mixture is stirred and a mixture consisting of 5.0 g of N-hexyl-1,8-naphthalimide-4-oxypropane acrylate (monomer of example 1) in 20.0 g of toluene is added.

0.5 g of Trigonox 21S (tert-butyl peroxy-2-ethyl-hexanoate) is added and the reaction mixture is then heated to 90° C.; the stirring and heating are continued for 6 hours and the mixture is then cooled to room temperature. The resulting polymer is purified by precipitation.

A statistical polymer comprising (weight %): 70% isobornyl acrylate, 20% ethylhexyl acrylate and 10% monomer according to the invention is obtained.

### EXAMPLE 11

A statistical copolymer comprising a monomer according to the invention is prepared. A mixture consisting of 15 g of N-hexyl-1,8-naphthalimide-4-oxypropane (monomer of example 1) in 55 g of toluene is placed in a reactor, under argon, equipped with a condenser and a stirrer, in the presence of 0.5 g of Trigonox 21S (t-butyl peroxy-2-ethylhexanoate). A homogeneous solution is obtained, to which are added 20 g of isobutyl methacrylate and 15 g of isobutyl acrylate, diluted in 5 g of toluene. The reaction mixture is heated to 90° C.; the stirring and heating are continued for 6 hours and the mixture is then cooled to room temperature.

After replacing the toluene with isododecane, a solution with a solids content of 50%, of polymer in isododecane, is obtained.

A statistical polymer comprising (weight %): 40% isobutyl methacrylate, 30% isobutyl acrylate and 30% monomer according to the invention is obtained.

The polymer has a weight-average molecular mass (Mw) of 63 000 and a number-average molecular mass (Mn) of 16 600, i.e. a polydispersity index Ip of 3.8.

### EXAMPLE 12

A statistical copolymer comprising a monomer according to the invention is prepared. A mixture consisting of 15 g of N-hexyl-1,8-naphthalimide-4-oxypropane (monomer of example 1) in 55 g of toluene is placed in a reactor, under argon, equipped with a condenser and a stirrer, in the presence of 0.5 g of Trigonox 21S (t-butyl peroxy-2-ethylhexanoate). A homogeneous solution is obtained, to which are added 10 g of isobutyl methacrylate and 7.5 g of isobutyl acrylate, diluted in 5 g of toluene. The reaction mixture is heated to 90° C.; the stirring and heating are continued for 6 hours and the mixture is then cooled to room temperature. The resulting polymer is purified by precipitation.

A statistical polymer comprising (weight %) 31% isobutyl methacrylate, 23% isobutyl acrylate and 46% monomer according to the invention is obtained.

### EXAMPLE 13

A statistical copolymer comprising a monomer according to the invention is prepared. A mixture consisting of 6 g of N-hexyl-1,8-naphthalimide-4-oxypropane (monomer of example 1) in 10 g of toluene is placed in a reactor, under argon, equipped with a condenser and a stirrer. A homogeneous solution is obtained, to which are added 14 g of 2-ethylhexyl acrylate, 0.4 g of Trigonox 21S (t-butyl peroxy-2-ethylhexanoate) and 15 g of isododecane. The reaction mixture is heated to 90° C.; the stirring and heating are continued for 7 hours and the mixture is then cooled to room temperature. The resulting polymer is purified by precipitation.

A statistical polymer comprising (weight %): 70% 2-ethylhexyl acrylate and 30% monomer according to the invention is obtained.

The polymer has a weight-average molecular mass (Mw) of 29 100 and a number-average molecular mass (Mn) of 8300, i.e. a polydispersity index Ip of 3.5.

### EXAMPLE 14

An anhydrous foundation is prepared, comprising (weight %):

| | |
|---|---|
| polyethylene wax | 12% |
| volatile silicone oils | 25% |
| phenyl trimethicone | 20% |
| polymethyl methacrylate microspheres | 12% |
| polymer of example 5 | 6% |
| isododecane | qs 100% |

Preparation:

The waxes are melted and, when the whole is clear, the phenyl trimethicone and the silicone oils are added with stirring; the microspheres, the isododecane and the polymer are then added. The mixture is homogenized for 15 minutes and the resulting composition is cast and allowed to cool.

An anhydrous foundation is obtained.

1. A cosmetic or pharmaceutical composition comprising, in a physiologically acceptable medium, at least one polymer comprising at least one monomeric compound of formula (I): 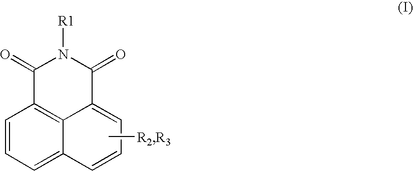
in which:
R1 represents a linear, branched and/or cyclic, saturated and/or unsaturated carbon-based radical containing 1 to 32 carbon atoms; optionally substituted with one or more groups chosen from ═O, OH, NH₂ and halogen atoms; and/or optionally interrupted with one or more heteroatoms chosen from O, N, P, Si and S;
R₂ and R₃, which are present on the same ring or each on a different ring, represent, independently of each other, a hydrogen, a halogen or a group of formula —X-G-P (II), with the proviso that at least one of the radicals R₂ and/or R₃ represents a group of formula (II), in which:
X is chosen from the groups —O—, —S—, —SO—, —SO₂—, —NH— and —NR₄— with R₄ representing a linear, branched and/or cyclic, saturated and/or unsaturated carbon-based radical containing 1 to 30 carbon atoms, optionally substituted with one or more groups chosen from ═O, OH, NH₂ and halogen atoms; and/or optionally interrupted with one or more heteroatoms chosen from O, N, P, Si and S;
G is a linear, branched and/or cyclic, saturated and/or unsaturated divalent carbon-based radical containing 1 to 32 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae: 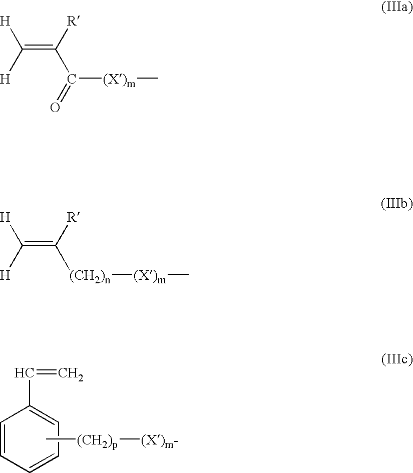
in which:
R′ represents H or a linear or branched, saturated C₁₋₆ hydrocarbon-based radical,
X′ represents O, NH or NR″ with R″ representing a radical chosen from C₁₋₆ alkyl, C₆₋₁₀ aryl, (C₆₋₁₀)-aryl(C₁₋₆)alkyl and (C₁₋₆)alkyl(C₆₋₁₀)aryl radicals, the alkyl and/or aryl groups also possibly being substituted with one or more groups chosen from OH, halogen, C₁₋₆ alkoxy and C₆₋₁₀ aryloxy;
and
m is equal to 0 or 1; n is equal to 0 or 1 and p is equal to 0, 1 or 2.

2. The composition as claimed in claim 1, in which, in the monomeric compound, R1 is a cyclic, linear and/or branched, saturated or unsaturated hydrocarbon-based radical, optionally comprising a hydrocarbon-based ring which is itself saturated or unsaturated, containing 3 to 18 and especially 4 to 14 carbon atoms, and may comprise at least one heteroatom, especially one or two nitrogen, oxygen or silicon atoms.

3. The composition as claimed in either of the preceding claims, in which, in the monomeric compound, R1 is a linear, branched and/or cyclic, saturated or unsaturated hydrocarbon-based radical containing 6 to 13 carbon atoms.

4. The composition as claimed in one of the preceding claims, in which, in the monomeric compound, R1 is chosen from n-propyl, isopropyl, n-butyl, isobutyl, tert-butyl, n-pentyl, cyclopentyl, n-hexyl, cyclohexyl, n-heptyl, n-octyl, cyclooctyl, decyl, cyclodecyl, dodecyl, cyclododecyl, p-tert-butylcyclohexyl, benzyl and phenyl radicals or a radical chosen from ethyl-2-N-pyrrolidine, (2-methyl)-1-ethylpyrrolidine, 3-propyl-triethoxysilane, dialkylpyrimidines and especially 2-(4,6-dimethyl)pyrimidine, the benzothiazyl radical and the fluorenyl radical.

5. The composition as claimed in one of the preceding claims, in which, in the monomeric compound, the radical R₂ is a hydrogen atom and R₃ is a group of formula (II).

6. The composition as claimed in one of the preceding claims, in which, in the monomeric compound, in the group of formula (II), X is chosen from —O—, —S—, —NH— and —NR₄—.

7. The composition as claimed in one of the preceding claims, in which, in the monomeric compound, the radical R₄ represents a linear, branched and/or cyclic, saturated or unsaturated hydrocarbon-based radical optionally comprising a hydrocarbon-based ring that is itself saturated or unsaturated, containing 2 to 18 and especially 3 to 12 carbon atoms, optionally substituted with one or more groups chosen from ═O, OH, NH₂ and halogen atoms; and/or optionally interrupted with one or more heteroatoms chosen from O, N, P, Si and S; especially an ethyl, n-propyl, isopropyl, n-butyl, isobutyl, tert-butyl, pentyl, hexyl, cyclohexyl, octyl, decyl, dodecyl, phenyl or benzyl radical.

8. The composition as claimed in one of the preceding claims, in which, in the monomeric compound, the divalent radical G is a linear, branched and/or cyclic, saturated or unsaturated divalent hydrocarbon-based radical optionally comprising a hydrocarbon-based ring that is itself saturated or unsaturated, containing in total 2 to 18 and especially 3 to 10 carbon atoms, optionally substituted with one or more groups chosen from ═O, OH, NH₂ and halogen atoms; and/or optionally interrupted with one or more heteroatoms chosen from O, N, P and Si.

9. The composition as claimed in one of the preceding claims, in which, in the monomeric compound, G is chosen from linear or branched, saturated divalent hydrocarbon-based radicals optionally comprising a saturated hydrocarbon-based ring, containing in total 2 to 18 and especially 3 to 10 carbon atoms.

10. The composition as claimed in one of the preceding claims, in which, in the monomeric compound, G is chosen from ethylene, n-propylene, isopropylene (or 1-methylethylene and 2-methylethylene), n-butylene, isobutylene, pentylene, especially n-pentylene, hexylene, especially n-hexylene, heptylene, cyclohexylene, octylene, decylene, cyclohexyl-dimethylene especially of formula —CH₂—C₆H₁₀—CH₂—, and dodecylene radicals.

11. The composition as claimed in one of the preceding claims, in which, in the monomeric compound, the polymerizable group P is chosen from one of the following formulae: 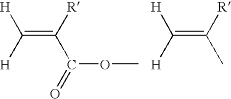
in which R′ represents H or methyl.

12. The composition as claimed in one of the preceding claims, in which the monomeric compound corresponds to one of the following formulae:

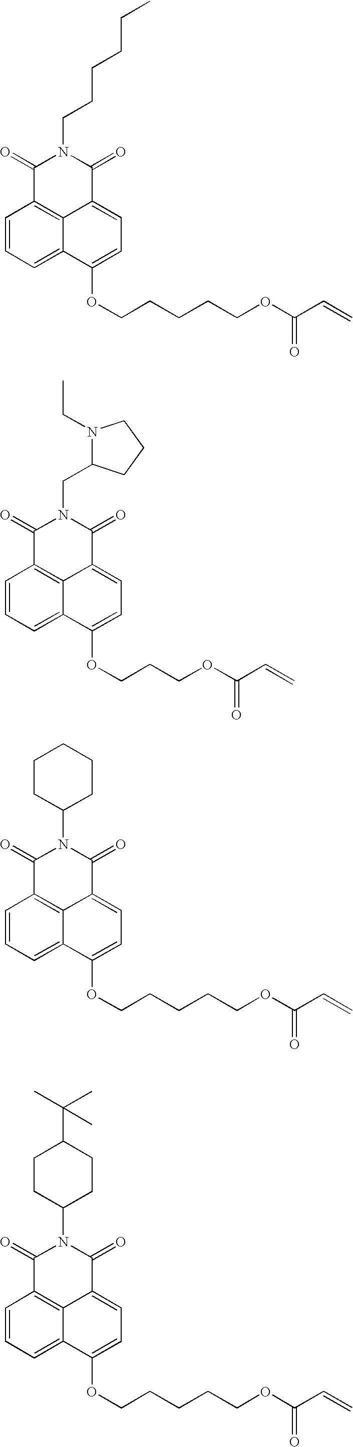

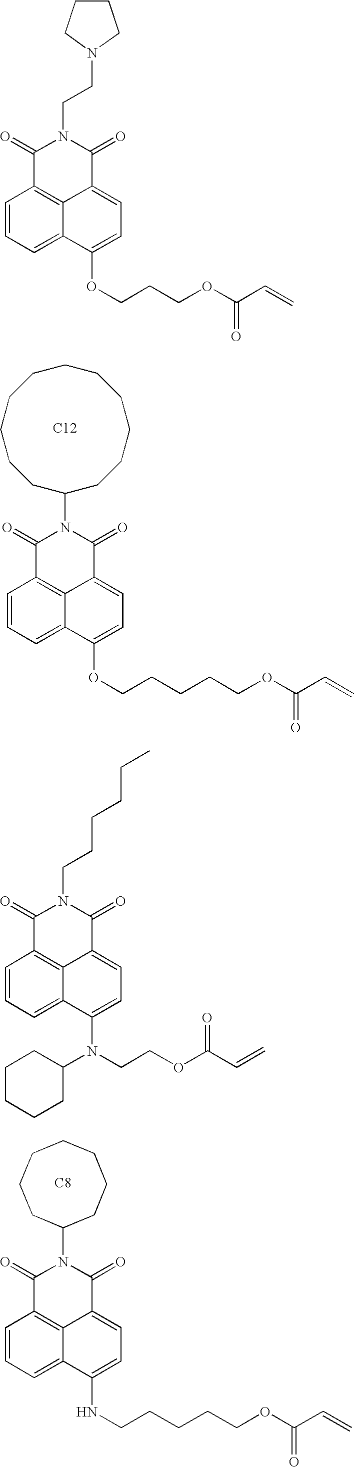

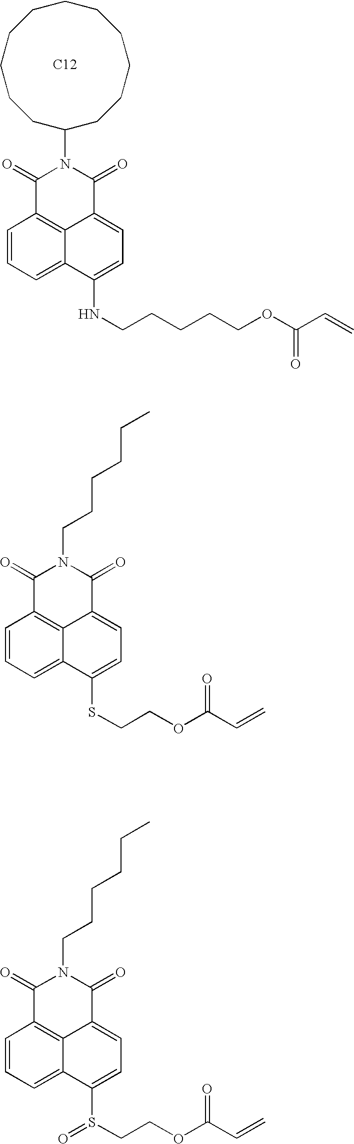

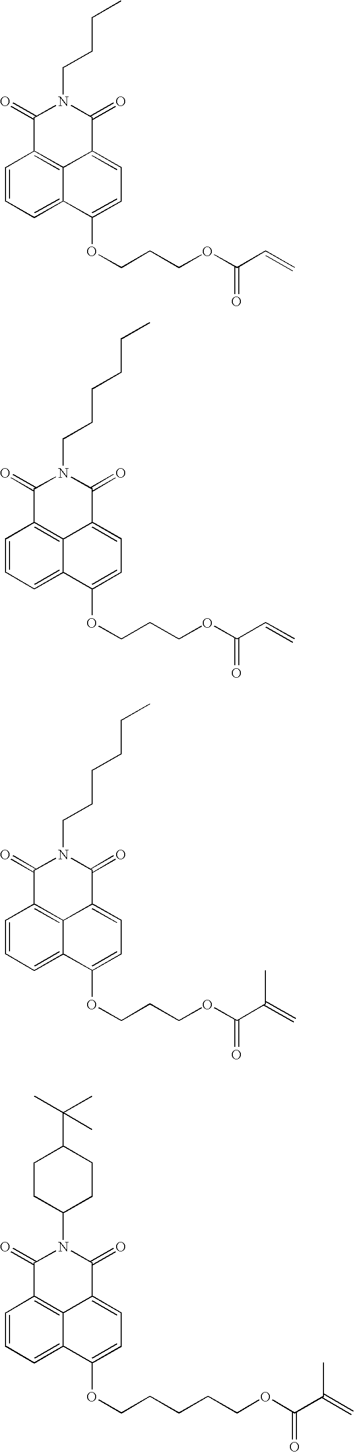

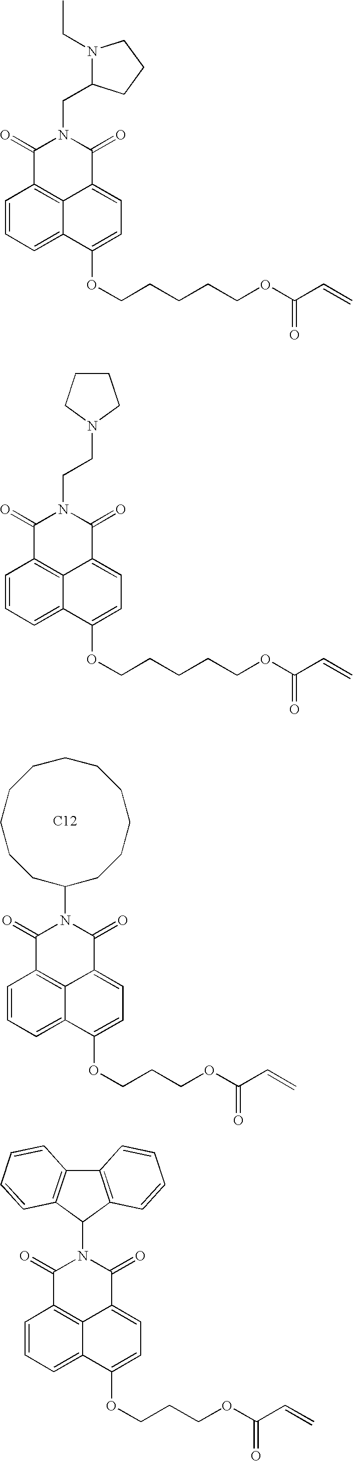

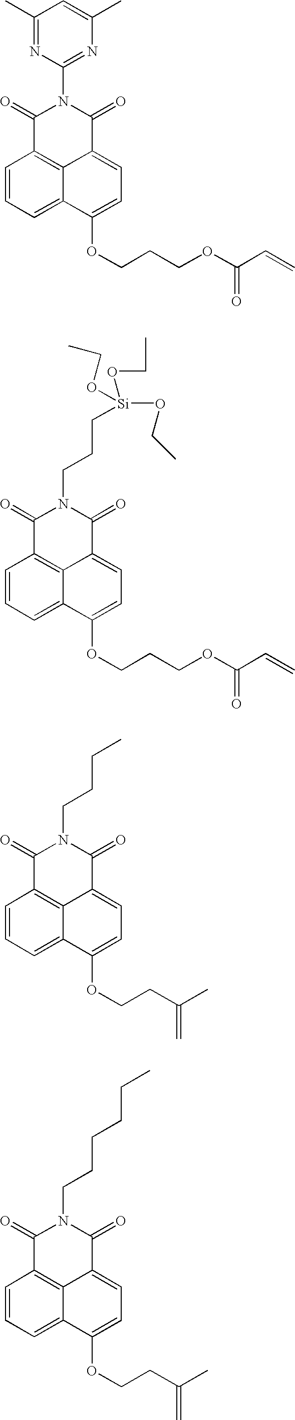

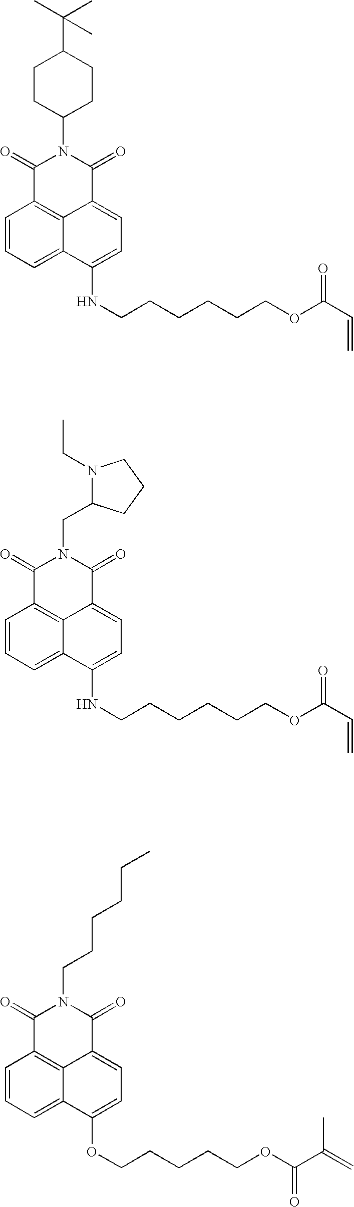

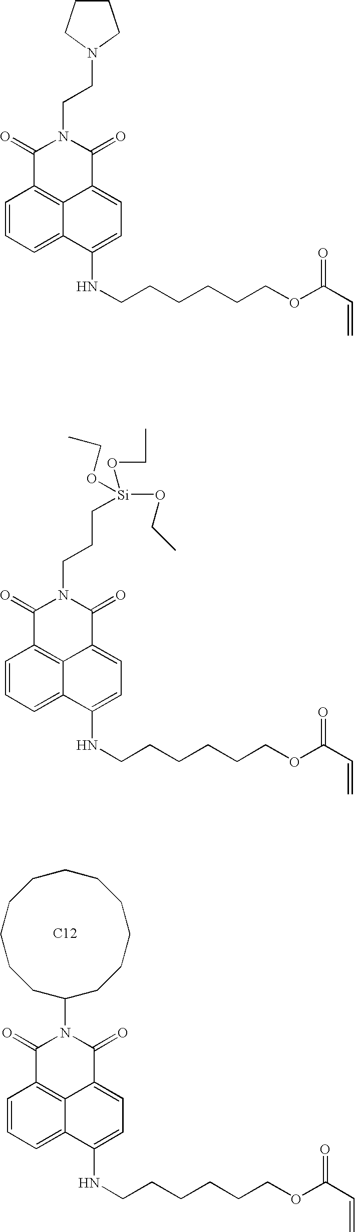

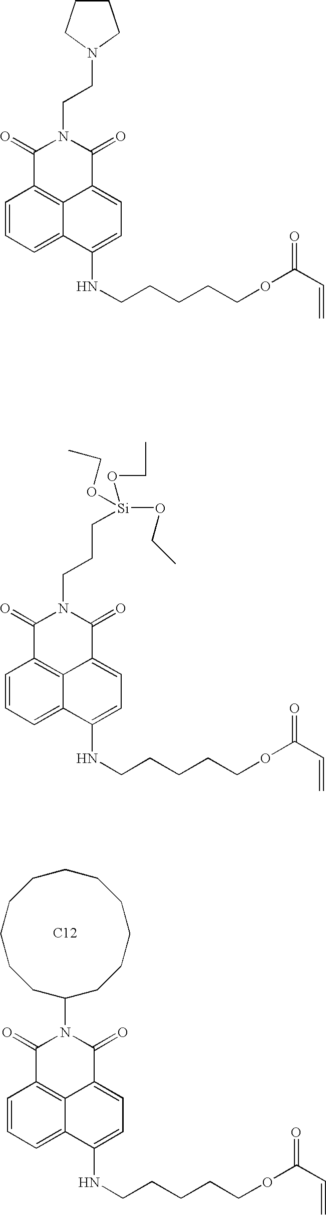

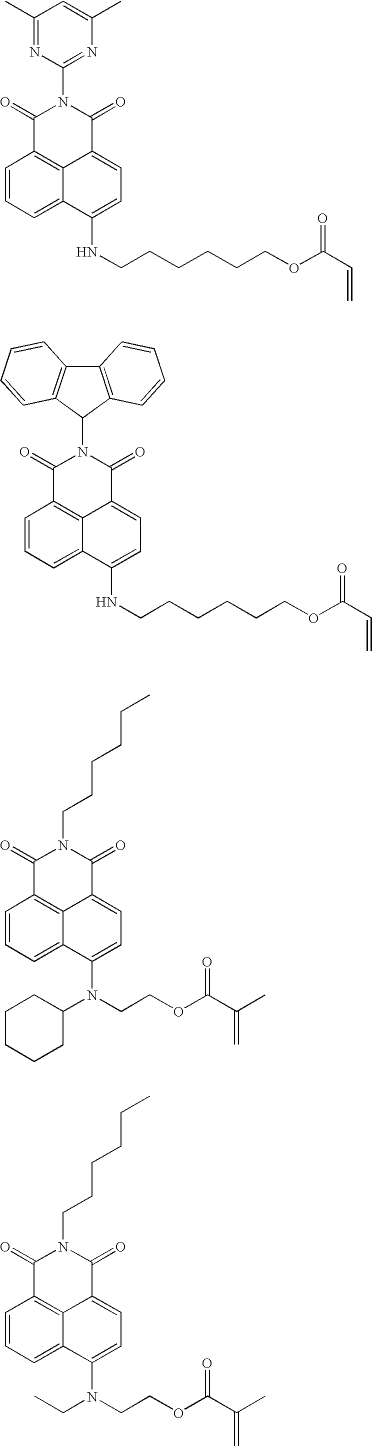

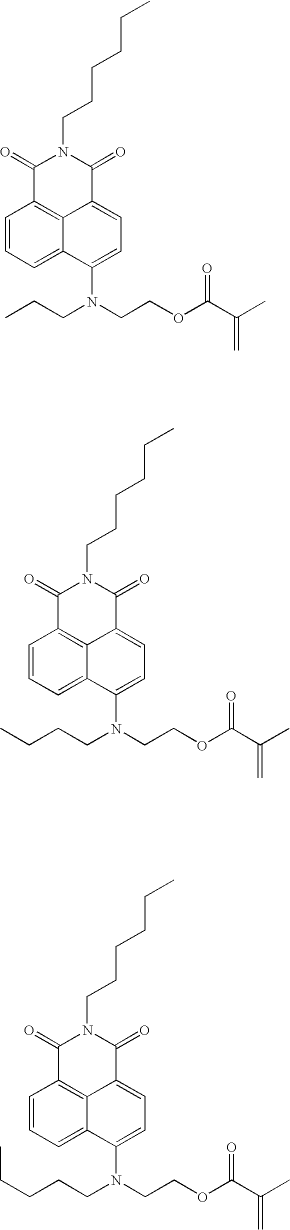

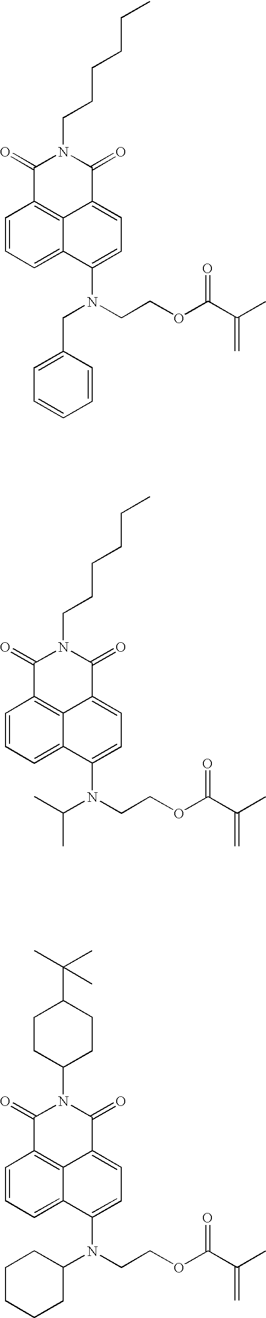

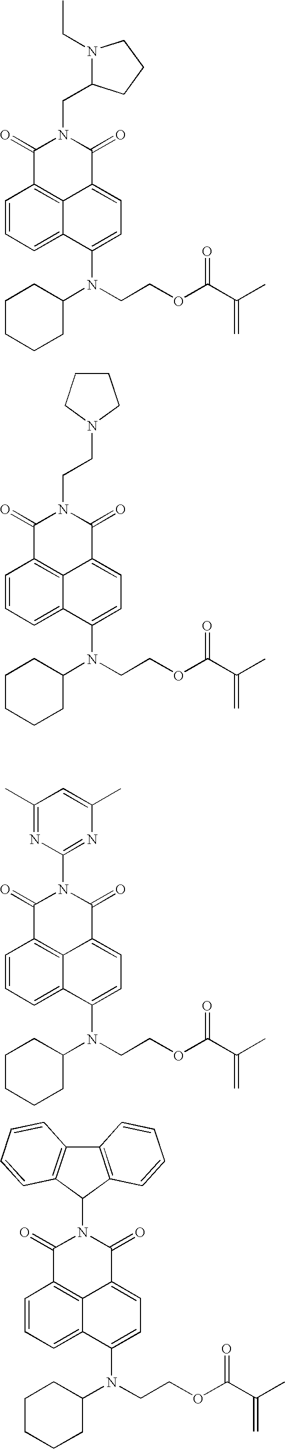

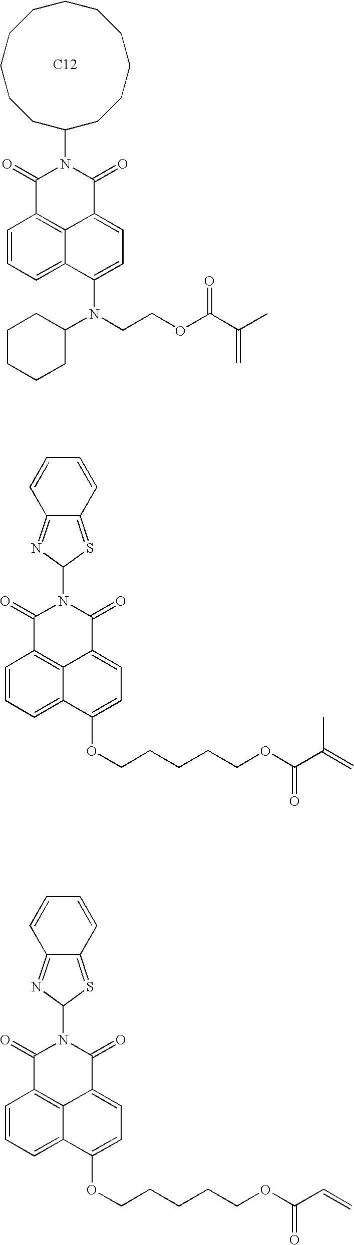

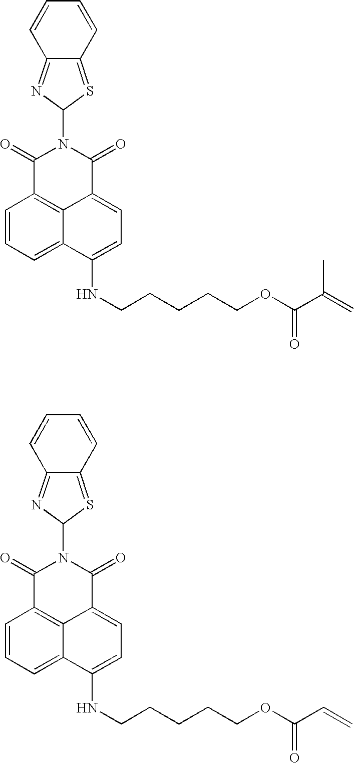

13. The composition as claimed in one of the preceding claims, in which the polymer is a homopolymer of a monomeric compound as defined in one of claims 1 to 12.

14. The composition as claimed in one of claims 1 to 12, in which the polymer is a copolymer comprising only monomeric compounds as defined in one of claims 1 to 12.

15. The composition as claimed in one of claims 1 to 12, in which the polymer is a copolymer comprising at least one monomeric compound as defined in one of claims 1 to 12, and at least one additional comonomer.

16. The composition as claimed in either of claims 14 and 15, in which the polymer is a statistical, alternating, grafted, block or gradient copolymer.

17. The composition as claimed in either of claims 15 and 16, in which the monomeric compound is present in an amount of from 0.01% to 70% by weight relative to the weight of said polymer, especially in an amount of from 0.1% to 50% by weight, in particular from 0.5% to 30% by weight, or even from 1% to 20% by weight and better still from 2% to 10% by weight, the additional comonomers, alone or as a mixture, representing the remainder to 100% by weight.

18. The composition as claimed in one of claims 15 to 17, in which the polymer comprises at least one additional comonomer chosen, alone or as a mixture, from the following monomers:
(i) ethylenic hydrocarbons containing from 2 to 10 carbons, such as ethylene, isoprene or butadiene;
(ii) the (meth)acrylates of formula:
CH₂═CHCOOR₃ or 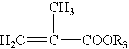
in which R₃ represents:
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F), and groups Si (R₄R₅), in which R₄ and R₅, which may be identical or different, represent a C₁ to C₆ alkyl group or a phenyl group;
R₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substitutents chosen from hydroxyl groups, halogen atoms and linear or branched C₁₋₄ alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F), and groups Si(R₄R₅), in which R₄ and R₅, which may be identical or different, represent a C₁ to C₆ alkyl group or a phenyl group,
R₃ may also be a group —(C₂H₄O)ₘ—R″, with m=5 to 150 and R″═H or C₁ to C₃₀ alkyl, for example —POE-methyl or —POE-behenyl;
(iii) the (meth)acrylamides of formula: 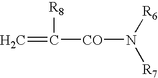
in which R₈ denotes H or methyl; and R₇ and R₆, which may be identical or different, represent:
a hydrogen atom; or
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F), and groups Si(R₄R₅), in which R₄ and R₅, which may be identical or different, represent a C₁ to C₆ alkyl group or a phenyl group;
R₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group, such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substitutents chosen from hydroxyl groups, halogen atoms and linear or branched C₁-C₄ alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F) and groups Si(R₄R₅), in which R₄ and R₅, which may be identical or different, represent a C₁ to C₆ alkyl group, or a phenyl group;
(iv) the vinyl compounds of formulae:
CH₂═CH—R₉, CH₂═CH—CH₂—R₉ or CH₂═C(CH₃)—CH₂—R₉
in which R₉ is a hydroxyl group, halogen (Cl or F), NH₂, OR₁₀ in which R₁₀ represents a phenyl group or a C₁ to C₁₂ alkyl group (the monomer is a vinyl or allylic ether); acetamide(NHCOCH₃); a group OCOR₁₁ in which R₁₁ represents a linear or branched alkyl group of 2 to 12 carbons (the monomer is a vinyl or allylic ester); or a group chosen from:
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F) and groups Si(R₄R₅), in which R₄ and R₅, which may be identical or different, represent a C₁ to C₆ alkyl group or a phenyl group;
a C₃ to C₁₂ cycloalkyl group such as isobornyl or cyclohexane,
a C₃ to C₂₀ aryl group such as phenyl,
a C₄ to C₃₀ aralkyl group (C₁ to C₈ alkyl group) such as 2-phenylethyl; benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substitutents chosen from hydroxyl groups, halogen atoms and linear or branched 1 to 4 C alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F) and groups Si(R₄R₅) in which R₁ and R₂, which may be identical or different, represent a C₁ to C₆ alkyl group, or a phenyl group;
(v) (meth)acrylic, (meth)acrylamide or vinyl monomers containing a fluoro or perfluoro group, such as ethylperfluorooctyl or 2-ethylperfluorohexyl (meth)acrylate;
(vi) silicone-based (meth)acrylic, (meth)acrylamide or vinyl monomers, such as methacryloxypropyltris(trimethylsiloxy)silane or acryloxypropylpolydimethylsiloxane;
(vii) ethylenically unsaturated monomers comprising at least one carboxylic, phosphoric or sulfonic acid, or anhydride, function, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;
(viii) ethylenically unsaturated monomers comprising at least one tertiary amine function, for instance 2-vinylpyridine, 4-vinylpyridine, dimethylaminoethyl methacrylate, diethylaminoethyl methacrylate or dimethylaminopropylmethacrylamide, and the salts thereof.

19. The composition as claimed in one of claims 15 to 18, in which the additional comonomer(s) is (are) present in an amount of from 30% to 99.99% by weight, especially in an amount of from 50% to 99.9% by weight, in particular from 70% to 99.5% by weight, or even from 80% to 99% by weight, and better still from 90% to 98% by weight, relative to the weight of the final polymer.

20. The composition as claimed in one of claims 15 to 19, in which the additional comonomers are chosen, alone or as a mixture, from C₁-C₁₈ alkyl or C₃-C₁₂ cycloalkyl(meth)acrylates, and especially from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate; or alternatively acrylic acid, methacrylic acid, methacryloxypropyltris(trimethylsiloxy)silane, acryl-oxypropyltris(trimethylsiloxy)silane, acryloxypropyl-polydimethylsiloxane and methacryloxypropylpoly-dimethylsiloxane.

21. The composition as claimed in one of claims 15 to 20, in which the polymer has a weight-average molecular mass (Mw) of between 5000 and 600 000 g/mol, especially between 10 000 and 300 000 g/mol and better still between 20 000 and 150 000 g/mol.

22. The composition as claimed in one of the preceding claims, in which the polymer is present, alone or as a mixture, in an amount of from 0.01% to 60% by weight, preferably 0.1% to 50% by weight, especially 1% to 25% by weight or even 3% to 15% by weight and better still 5% to 12% by weight, relative to the total weight of the composition.

23. The composition as claimed in one of the preceding claims, in which the physiologically acceptable medium comprises a hydrophilic medium comprising water or a water/hydrophilic organic solvent(s) mixture and/or comprises a fatty phase.

24. The composition as claimed in one of the preceding claims, in which the fatty phase comprises waxes, pasty fatty substances, gums, lipophilic organic solvents and oils, and/or mixtures thereof.

25. The composition as claimed in one of the preceding claims, also comprising a particulate phase that may comprise pigments and/or nacres and/or fillers.

26. The composition as claimed in one of the preceding claims, comprising dyestuffs chosen from water-soluble dyes and/or liposoluble dyes.

27. The composition as claimed in one of the preceding claims, comprising at least one additional polymer such as a film-forming polymer.

28. The composition as claimed in one of the preceding claims, comprising at least one ingredient chosen from vitamins, thickeners, gelling agents, trace elements, softeners, sequestrants, fragrances, acidifying or basifying agents, preserving agents, sunscreens, surfactants, antioxidants, hair-loss counteractants, antidandruff agents, propellants and ceramides, or mixtures thereof.

29. The composition as claimed in one of the preceding claims, which is in the form of a suspension, a dispersion especially of oil in water by means of vesicles; an optionally thickened or even gelled oily solution; an oil-in-water, water-in-oil or multiple emulsion; a gel or a mousse; an oily or emulsified gel;
a dispersion of vesicles, especially lipid vesicles; a two-phase or multi-phase lotion; a spray; a loose, compact or cast powder; an anhydrous pastep; a lotion, a cream, a pomade, a soft pastep, an ointment, a cast or molded solid especially as a stick or in a dish, or alternatively a compacted solid.

30. The composition as claimed in one of the preceding claims, which is in the form of a care and/or makeup product for bodily or facial skin, the lips, the nails, the eyelashes, the eyebrows and/or the hair, an antisun or self-tanning product, or a hair product for caring for, treating, shaping, making up or dyeing the hair.

31. The composition as claimed in one of the preceding claims, which is in the form of a makeup composition, especially a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer); a composition for protecting or caring for the skin of the face, the neck, the hands or the body, especially an antiwrinkle composition or a moisturizing or treating composition; an antisun or artificial tanning composition; a hair product, especially for dyeing, holding the hairstyle, shaping the hair, caring for, treating or cleansing the hair, such as shampoos, hairsetting gels or lotions, blow-drying lotions, and fixing and styling compositions such as lacquers or sprays.

32. A cosmetic process for making up or caring for keratin materials, especially bodily or facial skin, the lips, the nails, the eyelashes, the eyebrows and/or the hair, comprising the application to said materials of a cosmetic composition as defined in any one of claims 1 to 31.

33. A monomeric compound of formula (I): 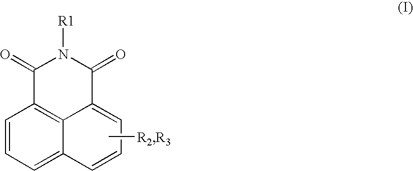
in which:
R1 represents a linear, branched and/or cyclic, saturated and/or unsaturated carbon-based radical, preferably alkyl, containing 2 to 22 carbon atoms and especially 8 to 16 carbon atoms; optionally substituted with one or more groups chosen from ═O, OH, NH₂ and halogen atoms; and/or optionally interrupted with one or more heteroatoms chosen from O, N, P, Si and S;
R₂ and R₃, present on the same ring or each on a different ring, represent, independently of each other, a hydrogen, a halogen or a group of formula —X-G-P (II), with the proviso that at least one of the radicals R₂ and/or R₃ represents a group of formula (II), in which:
X is chosen from —O—, —S—, —SO—, —SO₂—, —NH— and —NR₄— groups with R₄ representing a linear, branched and/or cyclic, saturated and/or unsaturated carbon-based radical containing 2 to 30 carbon atoms, optionally substituted with one or more groups chosen from ═O, OH, NH₂ and halogen atoms; and/or optionally interrupted with one or more heteroatoms chosen from O, N, P, Si and S;
G is a linear, branched and/or cyclic, saturated and/or unsaturated divalent carbon-based radical, containing 2 to 32 carbon atoms, optionally substituted with one or more groups chosen from ═O, OH, NH₂ and halogen atoms; and/or optionally interrupted with one or more heteroatoms chosen from O, N, P, Si and S;
P is a polymerizable group chosen from one of the following formulae: 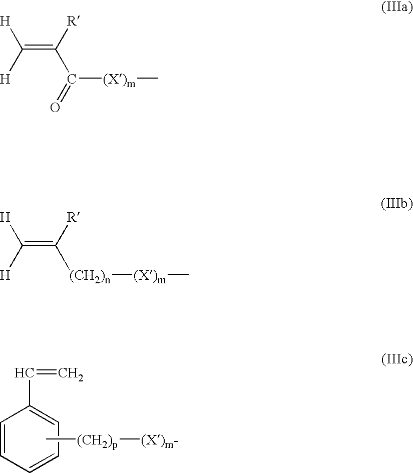
in which:
R′ represents H or a linear or branched, saturated C₁₋₆ hydrocarbon-based radical,
X′ represents O, NH or NR″ with R″ representing a radical chosen from C₁₋₆ alkyl, C₆₋₁₀ aryl, (C₆₋₁₀)-aryl(C₁₋₆)alkyl and (C₁₋₆)alkyl(C₆₋₁₀)aryl radicals, the alkyl and/or aryl groups also possibly being substituted with one or more groups chosen from OH, halogen, C₁₋₆ alkoxy and C₆₋₁₀ aryloxy;
m is equal to 0 or 1;
n is equal to 0 or 1;
p is equal to 0, 1 or 2,
with the exclusion of the compounds of formula (I) for which, simultaneously, R₂ is H, X is NH, P is of formula (IIIa), m=1, R′ is H or CH₃, X′ is O or NR″ and R1 is a C₁-C₆ alkyl radical, a C₆-C₁₀ aryl radical, a (C₆-C₁₀)aryl(C₁-C₆)alkyl radical or a (C₁-C₆)alkyl(C₆-C₁₀)aryl radical, said alkyl and/or aryl radicals being optionally substituted with a hydroxyl, a halogen, a (C₁-C₆)alkoxy or a (C₆-C₁₀)aryloxy.

34. The monomeric compound as claimed in claim 33, in which R1 is a cyclic, linear and/or branched, saturated or unsaturated hydrocarbon-based radical, optionally comprising a hydrocarbon-based ring which is itself saturated or unsaturated, containing 3 to 18 and especially 8 to 16 carbon atoms, and may comprise at least one heteroatom, especially one or two nitrogen, oxygen or silicon atoms.

35. The monomeric compound as claimed in either of claims 33 and 34, in which R1 is a linear, branched and/or cyclic, saturated or unsaturated hydrocarbon-based radical containing 8 to 16 carbon atoms.

36. The monomeric compound as claimed in claim 33, in which R1 is chosen from n-propyl, isopropyl, n-butyl, isobutyl, tert-butyl, n-pentyl, cyclopentyl, n-hexyl, cyclohexyl, n-heptyl, n-octyl, cyclooctyl, decyl, cyclodecyl, dodecyl, cyclododecyl, p-tert-butylcyclohexyl, benzyl and phenyl radicals or a radical chosen from ethyl-2-N-pyrrolidine, (2-methyl)-1-ethylpyrrolidine, 3-propyltriethoxysilane, dialkylpyrimidines and especially 2-(4,6-dimethyl)pyrimidine, the benzothiazyl radical and the fluorenyl radical.

37. The monomeric compound as claimed in one of claims 33 to 36, in which the radical R₂ is a hydrogen atom and R₃ is a group of formula (II).

38. The monomeric compound as claimed in one of claims 33 to 37, in which, in said group of formula (II), X is chosen from —O—, —S—, —NH— and —NR₄—.

39. The monomeric compound as claimed in one of claims 33 to 38, in which the radical R₄ represents a linear, branched and/or cyclic, saturated or unsaturated hydrocarbon-based radical optionally comprising a hydrocarbon-based ring that is itself saturated or unsaturated, containing 2 to 18 and especially 3 to 12 carbon atoms, optionally substituted with one or more groups chosen from ═O, OH, NH₂ and halogen atoms; and/or optionally interrupted with one or more heteroatoms chosen from O, N, P, Si and S; especially an ethyl, n-propyl, isopropyl, n-butyl, isobutyl, tert-butyl, pentyl, hexyl, cyclohexyl, octyl, decyl, dodecyl, phenyl or benzyl radical.

40. The monomeric compound as claimed in one of claims 33 to 39, in which the divalent radical G is a linear, branched and/or cyclic, saturated or unsaturated divalent hydrocarbon-based radical optionally comprising a hydrocarbon-based ring that is itself saturated or unsaturated, containing in total 2 to 18 and especially 3 to 10 carbon atoms, optionally substituted with one or more groups chosen from ═O, OH, NH₂ and halogen atoms; and/or optionally interrupted with one or more heteroatoms chosen from O, N, P and Si.

41. The monomeric compound as claimed in one of claims 33 to 40, in which G is chosen from linear or branched, saturated divalent hydrocarbon-based radicals optionally comprising a saturated hydrocarbon-based ring, containing in total 2 to 18 and especially 3 to 10 carbon atoms.

42. The monomeric compound as claimed in one of claims 33 to 41, in which G is chosen from ethylene, n-propylene, isopropylene (or 1-methylethylene and 2-methylethylene), n-butylene, isobutylene, pentylene, especially n-pentylene, hexylene, especially n-hexylene, heptylene, cyclohexylene, octylene, decylene, cyclohexyldimethylene especially of formula —CH₂—C₆H₁₀—CH₂—, and dodecylene radicals.

43. The monomeric compound as claimed in one of claims 33 to 42, in which the polymerizable group P is chosen from one of the following formulae: 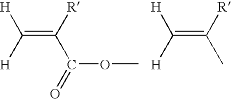
in which R′ represents H or methyl.

44. The monomeric compound as claimed in one of claims 33 to 43, corresponding to one of the following formulae:

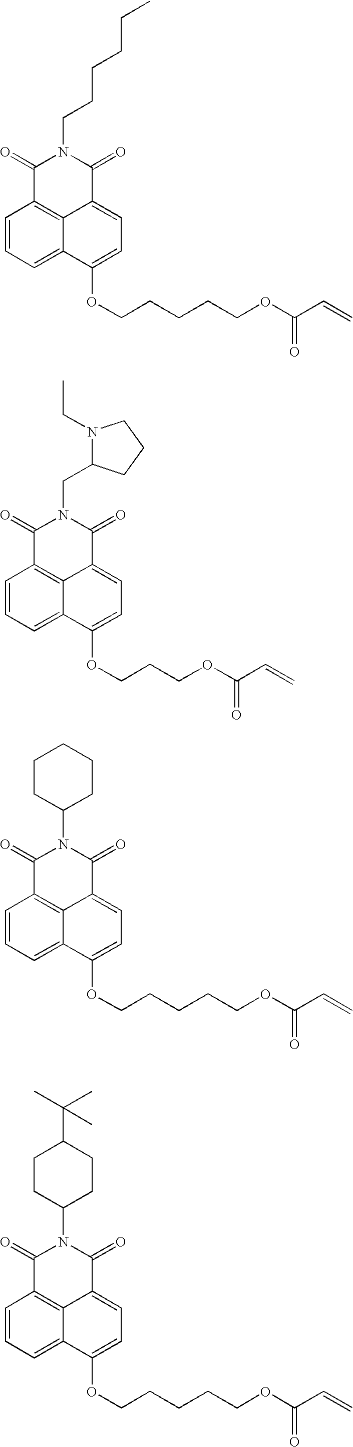

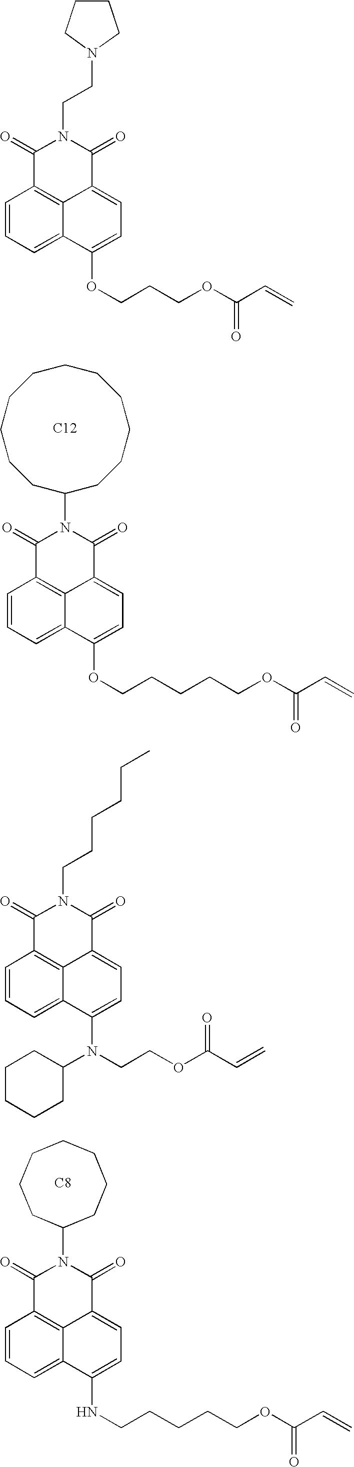

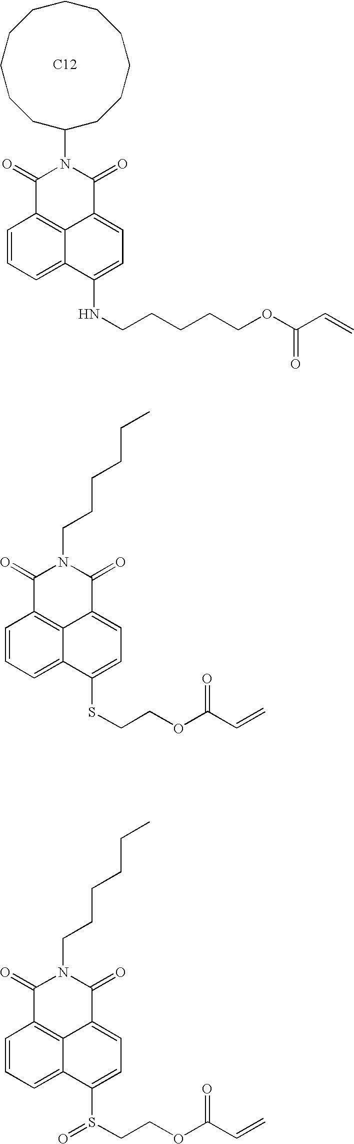

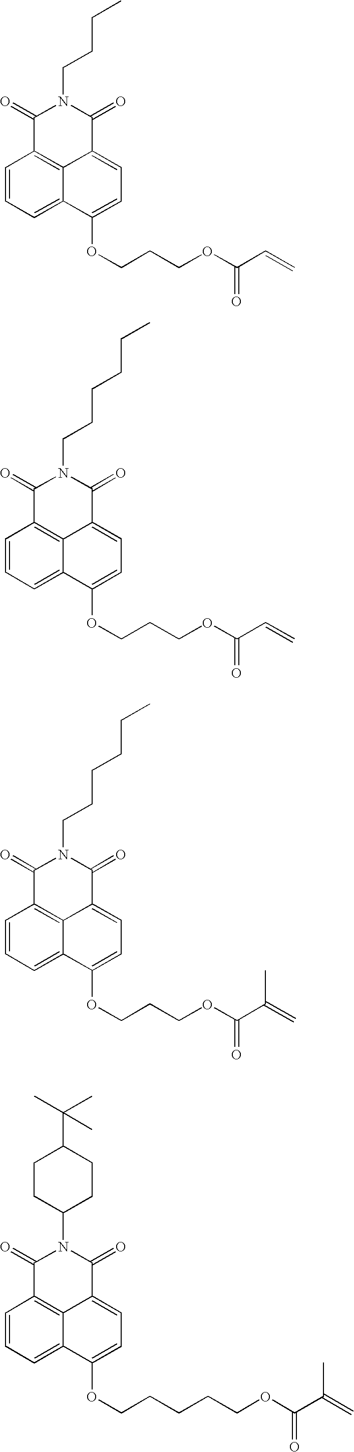

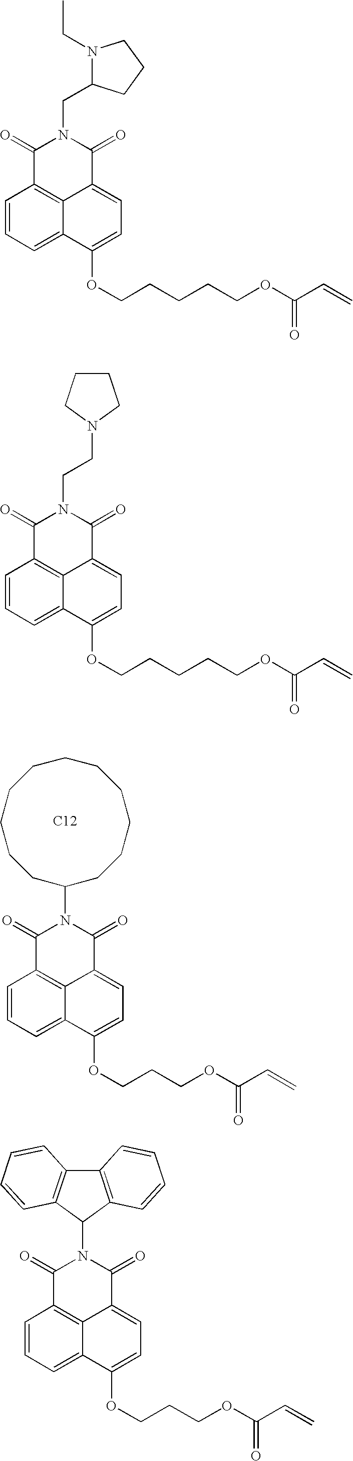

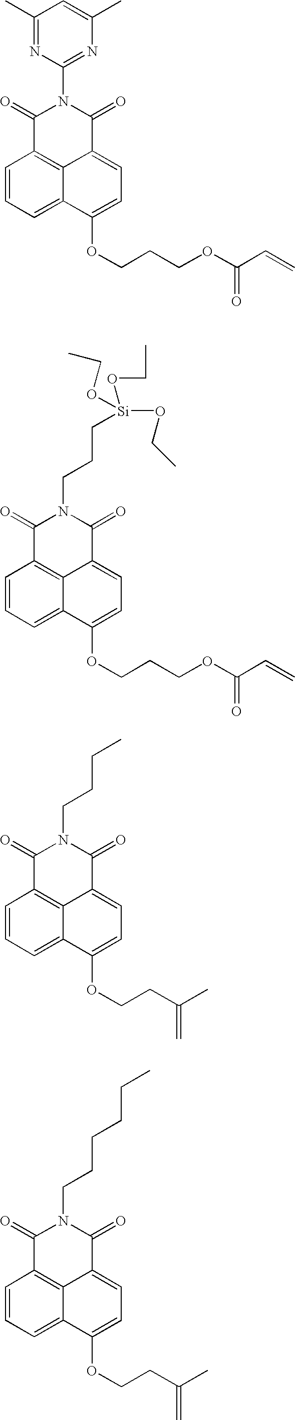

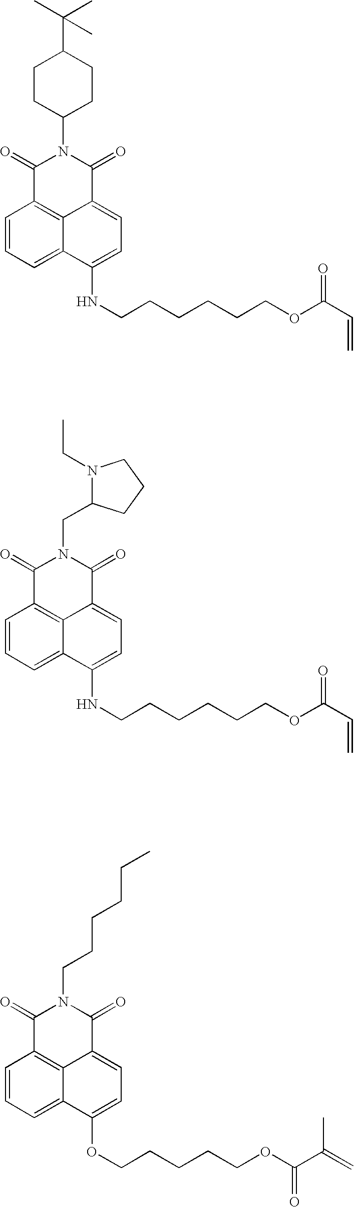

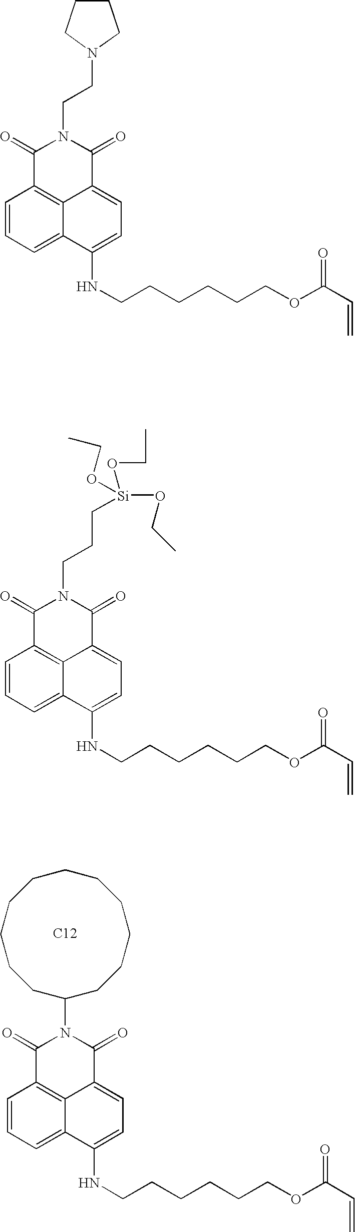

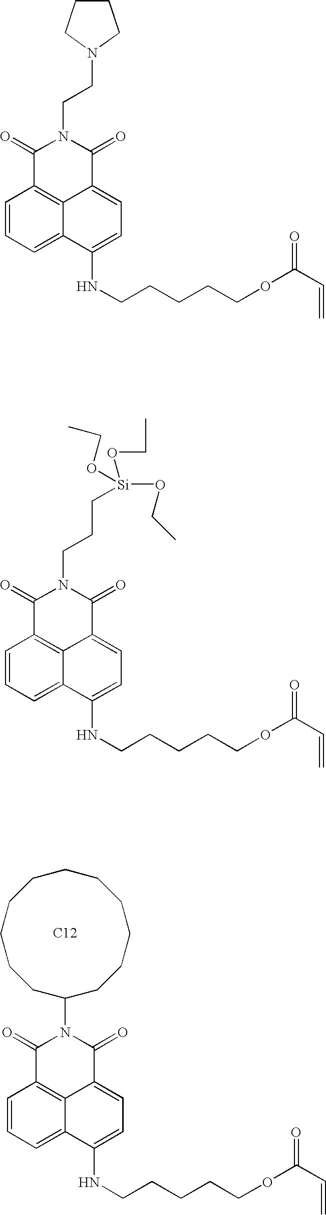

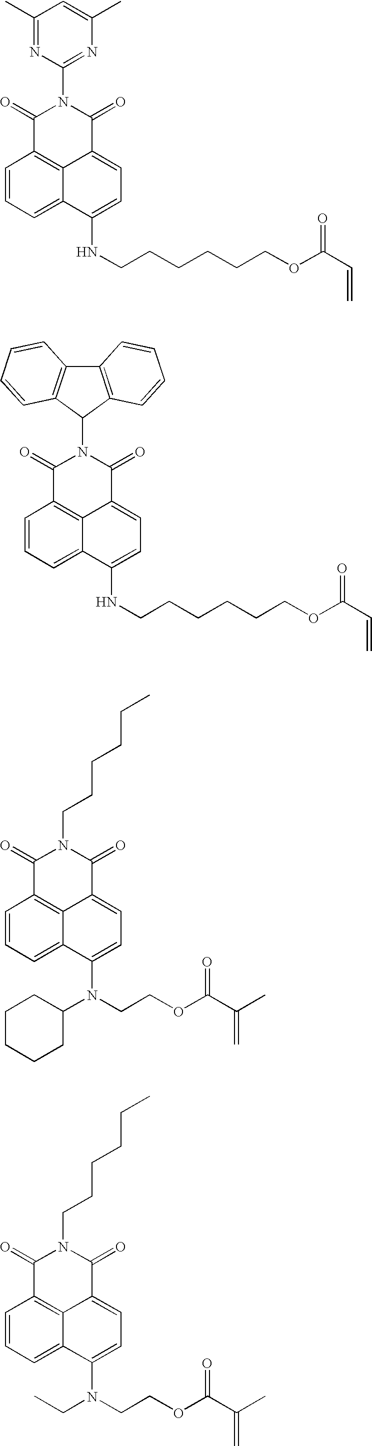

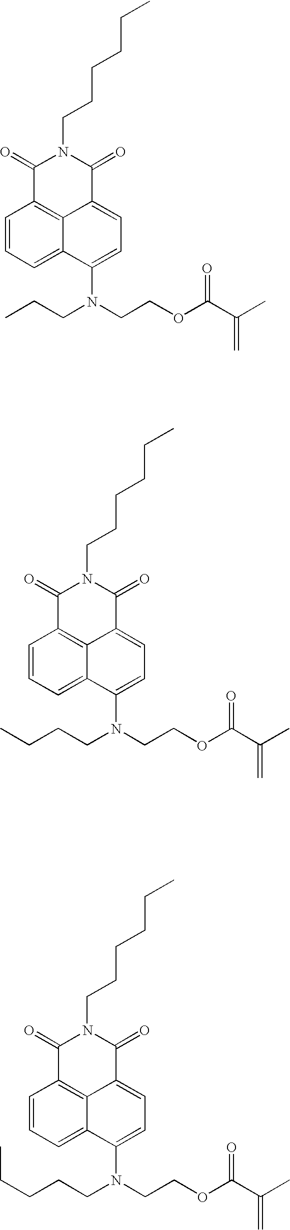

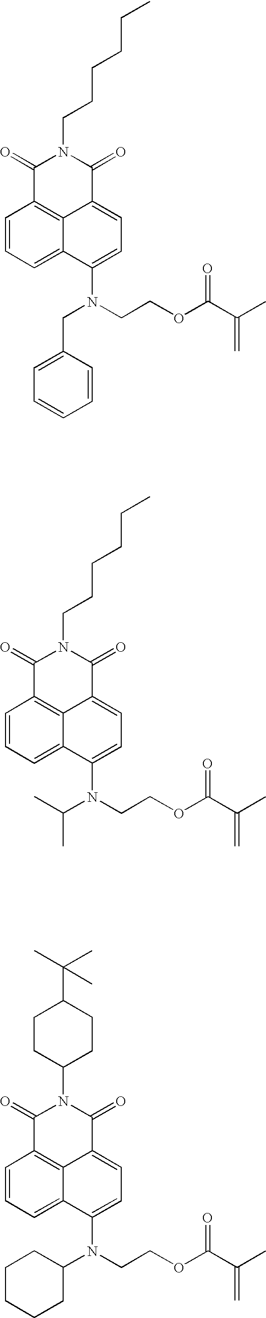

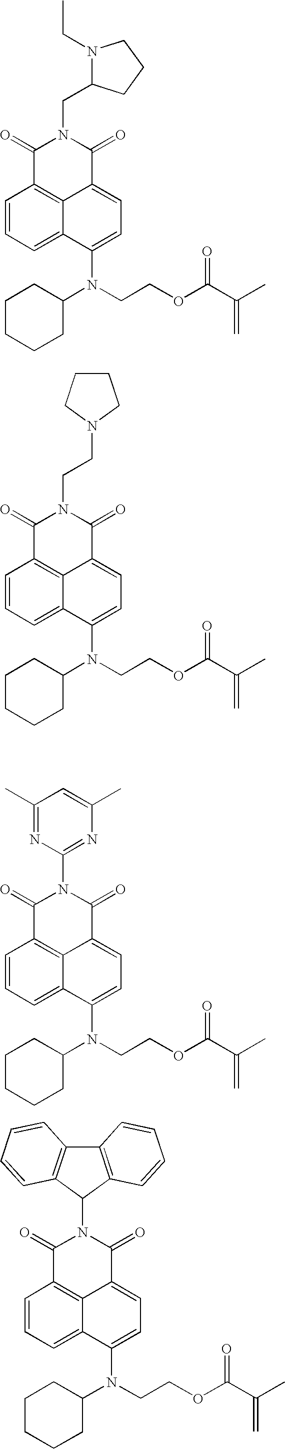

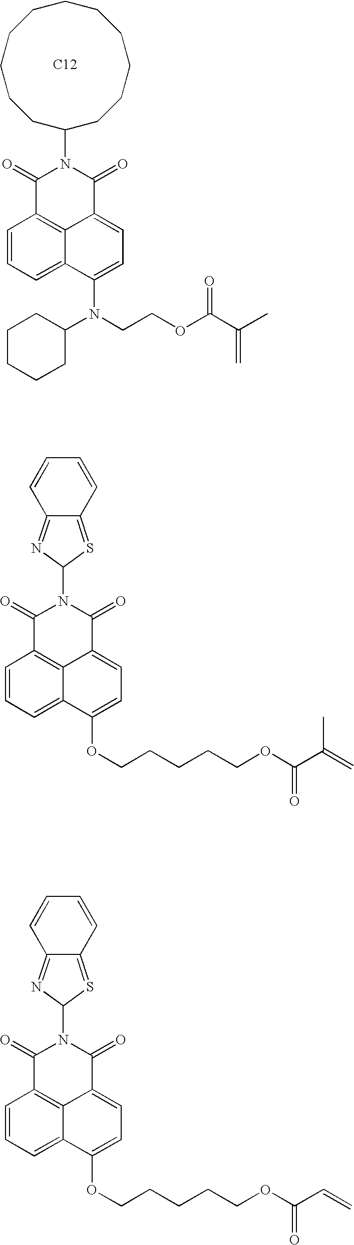

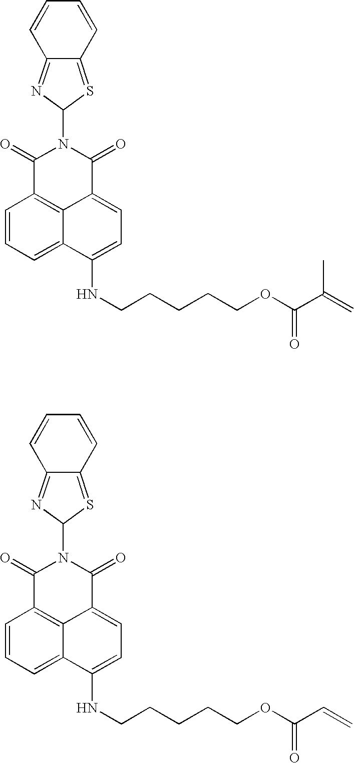

45. A polymer comprising at least one monomeric compound as defined in one of claims 33 to 44.

46. The polymer as claimed in claim 45, characterized in that it is a homopolymer of a monomeric compound as defined in one of claims 33 to 44.

47. The polymer as claimed in claim 45, characterized in that it is a copolymer comprising only monomeric compounds as defined in one of claims 33 to 44.

48. The polymer as claimed in claim 47, characterized in that the monomeric compounds are each present in a proportion of from 0.5% to 99.5% by weight, especially 5% to 95% by weight, or even 10% to 90% by weight and better still each in a proportion of from 30% to 70% by weight, relative to the total weight of the polymer.

49. The polymer as claimed in claim 45, characterized in that it is a copolymer comprising at least one monomeric compound as defined in one of claims 1 to 12, and at least one additional comonomer.

50. The polymer as claimed in claim 45, characterized in that it is a statistical, alternating, grafted, block or gradient copolymer.

51. The polymer as claimed in either of claims 49 and 50, characterized in that the monomeric compound is present in an amount of from 0.01% to 70% by weight relative to the weight of said polymer, especially in an amount of from 0.1% to 50% by weight, in particular from 0.5% to 30% by weight, or even from 1% to 20% by weight and better still from 2% to 10% by weight, the additional comonomers, alone or as a mixture, representing the remainder to 100% by weight.

52. The polymer as claimed in one of claims 49 to 51, characterized in that it comprises at least one additional hydrophilic comonomer, or a mixture of such comonomers, which may be present in a proportion of from 1% to 99.99% by weight, especially 2-70% by weight, better still 5-50% by weight or even 10-30% by weight, relative to the total weight of the copolymer.

53. The polymer as claimed in one of claims 49 to 51, characterized in that it comprises at least one additional hydrophobic comonomer, or a mixture of such comonomers, which may be present in a proportion of from 1% to 99.99% by weight, especially 30-98% by weight, better still 50-95% by weight or even 70-90% by weight, relative to the total weight of the copolymer.

54. The polymer as claimed in one of claims 49 to 51, characterized in that it comprises at least one additional comonomer chosen, alone or as a mixture, from the following monomers:
(i) ethylenic hydrocarbons containing from 2 to 10 carbons, such as ethylene, isoprene or butadiene;
(ii) the (meth)acrylates of formula:
CH₂═CHCOOR₃ or 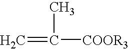
in which R₃ represents:
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F), and groups Si(R₄R₅), in which R₄ and R₅, which may be identical or different, represent a C₁ to C₆ alkyl group or a phenyl group;
R₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substitutents chosen from hydroxyl groups, halogen atoms and linear or branched C₁₋₄ alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F), and groups Si (R₄R₅), in which R₄ and R₅, which may be identical or different, represent a C₁ to C₆ alkyl group or a phenyl group,
R₃ may also be a group —(C₂H₄O)ₘ—R″, with m=5 to 150 and R″═H or C₁ to C₃₀ alkyl, for example —POE-methyl or —POE-behenyl;
(iii) the (meth)acrylamides of formula: 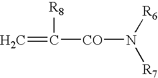
in which R₈ denotes H or methyl; and R₇ and R₆, which may be identical or different, represent:
a hydrogen atom; or
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F), and groups Si (R₄R₅), in which R₄ and R₅, which may be identical or different, represent a C₁ to C₆ alkyl group or a phenyl group;
R₃ may especially be a methyl, ethyl, propyl, n-butyl, isobutyl, tert-butyl, hexyl, ethylhexyl, octyl, lauryl, isooctyl, isodecyl, dodecyl, cyclohexyl, t-butyl-cyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,
a C₃ to C₁₂ cycloalkyl group, such as an isobornyl group,
a C₃ to C₂₀ aryl group such as a phenyl group,
a C₄ to C₃₀ aralkyl group (C₁ to C₈ alkyl group) such as 2-phenylethyl, t-butylbenzyl or benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substitutents chosen from hydroxyl groups, halogen atoms and linear or branched C₁-C₄ alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F) and groups Si (R₄R₅), in which R₄ and R₅, which may be identical or different, represent a C₁ to C₆ alkyl group, or a phenyl group;
(iv) the vinyl compounds of formulae:
CH₂═CH—R₉, CH₂═CH—CH₂—R₉ or CH₂═C(CH₃)—CH₂—R₉
in which R₉ is a hydroxyl group, halogen (Cl or F), NH₂, OR₁₀ in which R₁₀ represents a phenyl group or a C₁ to C₁₂ alkyl group (the monomer is a vinyl or allylic ether); acetamide(NHCOCH₃); a group OCOR₁₁ in which R₁₁ represents a linear or branched alkyl group of 2 to 12 carbons (the monomer is a vinyl or allylic ester); or a group chosen from:
a linear or branched alkyl group of 1 to 18 carbon atoms, in which is (are) optionally intercalated one or more heteroatoms chosen from O, N, S and P; said alkyl group also possibly being optionally substituted with one or more substitutents chosen from hydroxyl groups, halogen atoms (Cl, Br, I and F) and groups Si(R₄R₅), in which R₄ and R₅, which may be identical or different, represent a C₁ to C₆ alkyl group or a phenyl group;
a C₃ to C₁₂ cycloalkyl group such as isobornyl or cyclohexane,
a C₃ to C₂₀ aryl group such as phenyl,
a C₄ to C₃₀ aralkyl group (C₁ to C₈ alkyl group) such as 2-phenylethyl; benzyl,
a 4- to 12-membered heterocyclic group containing one or more heteroatoms chosen from O, N and S, the ring being aromatic or non-aromatic,
a heterocycloalkyl group (1 to 4 C alkyl), such as furfurylmethyl or tetrahydrofurfurylmethyl,
said cycloalkyl, aryl, aralkyl, heterocyclic or heterocycloalkyl groups possibly being optionally substituted with one or more substitutents chosen from hydroxyl groups, halogen atoms and linear or branched 1 to 4 C alkyl groups in which is (are) optionally intercalated one or more heteroatoms chosen from O, N, S and P, said alkyl groups also possibly being optionally substituted with one or more substitutents chosen from hydroxyl groups, halogen atoms (Cl, Br, I and F) and groups Si(R₄R₅) in which R₁ and R₂, which may be identical or different, represent a C₁ to C₆ alkyl group, or a phenyl group;
(v) (meth)acrylic, (meth)acrylamide or vinyl monomers containing a fluoro or perfluoro group, such as ethylperfluorooctyl or 2-ethylperfluorohexyl (meth) acrylate;
(vi) silicone-based (meth)acrylic, (meth)acrylamide or vinyl monomers, such as methacryloxypropyltris(trimethylsiloxy)silane or acryloxypropylpolydimethylsiloxane;
(vii) ethylenically unsaturated monomers comprising at least one carboxylic, phosphoric or sulfonic acid, or anhydride, function, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;
(viii) ethylenically unsaturated monomers comprising at least one tertiary amine function, for instance 2-vinylpyridine, 4-vinylpyridine, dimethylaminoethyl methacrylate, diethylaminoethyl methacrylate or dimethylaminopropylmethacrylamide, and the salts thereof.

55. The polymer as claimed in claim 54, characterized in that the additional comonomer(s) is (are) present in an amount of from 30% to 99.99% by weight, especially in an amount of from 50% to 99.9% by weight, in particular from 70% to 99.5% by weight or even from 80% to 99% by weight and better still from 90% to 98% by weight relative to the weight of the final polymer.

56. The polymer as claimed in one of claims 49-51 and 54-55, characterized in that the additional comonomers are chosen, alone or as a mixture, from C₁-C₁₈ alkyl or C₃-C₁₂ cycloalkyl(meth)acrylates, and especially from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate; or alternatively acrylic acid, methacrylic acid, methacryloxypropyltris(trimethyl-siloxy)silane, acryloxypropyltris(trimethylsiloxy)-silane, acryloxypropylpolydimethylsiloxane and methacryloxypropylpolydimethylsiloxane.

57. The polymer as claimed in one of claims 49 to 56, characterized in that it has a weight-average molecular mass (Mw) of between 5000 and 600 000 g/mol, especially between 10 000 and 300 000 g/mol and better still between 20 000 and 150 000 g/mol.

58. The use of at least one monomeric compound as defined in one of claims 33 to 44, or of at least one polymer as defined in one of claims 45 to 57, in a composition, for giving said composition optical effects, especially fluorescence or optical-brightening effects.
